(19) United States
(12) Patent Application Publication
(10) Pub. No.: US 2022/0347323 A1
(43) Pub. Date: Nov. 3, 2022

(54) Positron Emission Tomography Guided Delivery of Mitochondral Complex I Inhibitors

(71) Applicant: The Regents of the University of California, Oakland, CA (US)

(72) Inventors: David Shackelford, Rancho Palos Verdes, CA (US); Saman Sadeghi, Sherman Oaks, CA (US); Milica Momcilovic, Los Angeles, CA (US)

(21) Appl. No.: 17/760,846

(22) PCT Filed: Sep. 18, 2020
(86) PCT No.: PCT/US2020/051587

§ 371 Date: Mar. 16, 2022

Related U.S. Application Data
(60) Provisional application No. 62/901,947, filed on Sep. 18, 2019.

(51) Int. Cl. A61K 51/04; A61B 6/03; A61B 6/00; A61K 31/155; A61K 31/352; A61K 31/454; A61P 35/00; A61P 11/00

(52) U.S. Cl. CPC A61K 51/04; A61B 6/037; A61B 6/032; A61B 6/50; A61B 6/5235; A61B 6/5247; A61K 31/155; A61K 51/0491; A61K 31/352; A61K 31/454; A61P 35/00; A61P 11/00

(57) ABSTRACT

A method for detecting or ruling out non-small cell lung cancer (NS-CLC) in a patient comprises: (a) administering to a patient a detectable amount of a compound of formula (I): Formula (I) wherein the compound is targeted to any NSCLC tumor in the patient; and (b) acquiring an image to detect the presence or absence of any NSCLC tumor in the patient, wherein at least one of the atoms in formula (I) is replaced with ¹¹C, ¹³N, ¹⁵O, ¹⁸F, ^{34m}CI, ³⁸K, ⁴⁵Ti, ⁵¹Mn, ⁵²Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

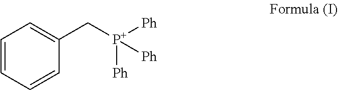 [SMILES: c1ccc(C[P+](c2ccccc2)(c2ccccc2)c2ccccc2)cc1]

### CROSS-REFERENCES TO RELATED APPLICATIONS

This application claims priority to U.S. Provisional Application No. 62/901,947, filed on Sep. 18, 2019, the disclosure of which is hereby incorporated by reference in its entirety.

### STATEMENT REGARDING FEDERALLY SPONSORED RESEARCH

This work was supported by the National Institutes of Health/National Cancer Institute through grant number R01-CA208642. The government has certain rights in the invention.

### BACKGROUND OF THE INVENTION

### 1. Field of the Invention

The invention relates to guided delivery of mitochondrial complex I inhibitors via imaging agents for positron emission tomography.

### 2. Description of the Related Art

Millions of people in the United States and all over the world are affected by cancers. Decades of research in both industry and academia have failed in many cases to develop highly effective treatments. One of the main reasons is that the available therapies do not effectively target the cancer. Investigation in mitochondrial cancer cells has provided new insight into the role of mitochondria in cancer.

The mitochondria are essential regulators of cellular energy and metabolism and they play a critical role in sustaining growth and survival of cancer cells. A central process of the mitochondria is the synthesis of ATP through oxidative phosphorylation (OXPHOS) known as bioenergetics. The mitochondria maintain OXPHOS by creating a membrane potential gradient (ΔΨ) that is generated by the electron transport chain (ETC) in order to drive ATP synthesis [Ref. 1,2]. Mitochondria are essential for tumor initiation and maintenance. However, little is known about oxidative mitochondrial metabolism in cancer because most studies have been performed in vitro in cell culture models.

Therefore, there is a large gap in the art on how oxidative mitochondrial metabolism supports tumor growth and highlights a need for in vivo studies.

Therefore, there exists a need for a tracer that measures the mitochondrial activity in cancer.

### SUMMARY OF THE INVENTION

To accelerate the understanding of mitochondrial activity in cancer, we have developed an imaging methodology to quantify mitochondrial ΔΨ in vivo using a voltage sensitive, positron emission tomography (PET) tracer. The development of the PET methodology allows to both visualize cancer tumors, specifically lung tumors, and measure the effects of therapeutic drug treatment.

The development of a PET methodology also allows to measure the mitochondrial ΔΨ in autochthonous models of lung cancer and revealed distinct functional mitochondrial heterogeneity within NSCLC tumor subtypes.

These and other features, aspects, and advantages of the present invention will become better understood upon consideration of the following detailed description, drawings, and appended claims.

### BRIEF DESCRIPTION OF THE DRAWINGS

FIG. 1 shows ¹⁸FBnTP PET imaging and biodistribution analysis of Kras/Lkb1 lung tumors identified differential uptake between lung adenocarcinomas (ADC) and squamous cell carcinomas (SCC). a, PET/CT overlay of a Kras/Lkb1 mouse with lung tumors, imaged with ¹⁸FBnTP probe. Right panel is rotated 90° compared to left panel. Heart and Tumor are indicated by arrows. L—liver; GI—gastrointestinal tract; K—Kidney; B—Bladder. b, Biodistribution of ¹⁸FBnTP probe in tissue from wild type FVB mice measured by gamma counter ex vivo after 1 hr uptake (n=5 mice). c, Biodistribution of the ¹⁸FBnTP probe in normal tissue of Kras/Lkb1 mice measured by % injected dose/gram after 1 hr uptake (n=12 mice). d, ¹⁸FBnTP uptake in lung ADC and SCC from Kras/Lkb1 mice (n=5 mice, n=10 ADC tumors, n=7 SCC tumors). e, Representative transverse image of the heart and lungs of a Kras/Lkb1 mouse imaged with CT (left panel) and ¹⁸FBnTP (right panel). H—heart, T1—adenocarcinoma (ADC), T2—squamous cell carcinoma (SCC). f, IHC staining of T1 and T2 tumors from panel e. TTF1—thyroid transcription factor 1; CK5—keratin 5; Tom20—translocase of outer membrane 20. Scale bar=100 μm. The data are represented as the mean +/− SD. Statistical significance (**p<0.01) was calculated using unpaired two-tailed t-test.

FIG. 2 demonstrates that treatment of Kras/Lkb1 GEMMS with the complex I inhibitor phenformin suppresses ¹⁸FBnTP uptake in lung tumors. a, Schematic drawing representing voltage dependent uptake of ¹⁸FBnTP into the mitochondria. Bioenergetics driven by electron transport chain (ETC), and oxidative phosphorylation (OXPHOS) are shown. b, TMRE measurements in A549 cells treated with indicated concentrations of Phenformin or FCCP for 3 hr (n=3 replicates). c, Uptake of ¹⁸FBnTP measured by gamma counter in A549 cells treated with 1 mM Phenformin for 3 hr (n=5 replicates). d, Oxygen consumption rate (OCR) per cell measured in A549 cells treated acutely with 1 mM Phenformin for 45 min (n=25 technical replicates). e, TMRE measurements in mouse cell line L3161C treated with indicated concentrations of Phenformin or FCCP for 3 hr (n=3 replicates). f, OCR per cell measured in mouse tumor line L3161C treated acutely with 1 mM Phenformin for 45 min (n=25 technical replicates). g, TMRE measurements in mouse cell line L3161C treated with Vehicle, 8 μM Oligomycin, or 8 μM Oligomycin+4 μM FCCP for 3 hr (n=3 replicates). h, Uptake of ¹⁸FBnTP probe measured by gamma counter in mouse cell line L3161C treated with Vehicle, 8 μM Oligomycin, or 8 μM Oligomycin+4 μM FCCP for 3 hr (n=6 replicates). i, Schematic drawing of imaging and treatment regiments for Kras/Lkb1 mice treated with 125 mg/kg/day Phenformin. j, Representative images from a Kras/Lkb1 mouse before (top panel) and after (bottom panel) treatment for 5 days with 125 mg/kg/day Phenformin. Values for maximum percent injected dose (%ID/g) for the heart and tumor are indicated. H—heart. k, Quantification of maximum percent injected dose (%ID/g) for tumors in Vehicle (Veh) and Phenformin (Phen) groups before treatment (Pre-treatment). Each dot represents an individual tumor; n=11 tumors for Vehicle and Phenformin groups. l, Quantification of ¹⁸FBnTP uptake for tumors in Veh and Phen groups after treatment (Post-treatment) with 125 mg/kg/day Phenformin for 5 days. Each dot represents individual tumor; n=11 tumors for Vehicle and Phenformin groups. m, Waterfall plot for % change in probe uptake post-treatment to pre-treatment for mice treated with Veh or Phen for 5 days. Each bar represents an individual tumor. n, Average ¹⁸FBnTP uptake in Kras/Lkb1 mice after 5 day treatment with Vehicle (Veh) (n=48 tumors; 10 mice) or Phenformin (Phen) (n=23 tumors; 10 mice). Each dot represents an individual tumor. The data are represented as the mean +/− SD. Statistical significance (*p<0.05; **p<0.01; ***p<0.001; ****p<0.0001; ns, not significant) was calculated using unpaired two-tailed t-test or one-way ANOVA (for b, e, g, h). Experiments in b-g were performed twice; experiment in h was performed once.

FIG. 3 demonstrates that ¹⁸FBnTP detects mitochondrial complex I inhibition in vivo. a, Schematic drawing of the transthoracic (TT) implantation of KPL lung adenocarcinoma (ADC) cells into syngeneic recipient mouse. b, Representative ¹⁸FBnTP PET/CT overlay of a tumor formed by transthoracically implanted L3161C cell line. H—heart, T—tumor. c, H&E slide of a tumor formed by transthoracically implanted L3161C cell line. Scale bar=2 mm. d, Schematic drawing of the treatment and imaging regiment for syngeneic mice implanted transthoracically (TT) with L3161C cell line and treated with a single dose of Vehicle or Oligomycin or Rotenone. e, Waterfall plot for % change in ¹⁸FBnTP uptake post-treatment to pre-treatment for mice treated with a single dose of Vehicle (n=7 mice) or 0.25 mg/kg Oligomycin (n=9 mice) or 0.5 mg/kg Rotenone (n=7 mice). Statistical significance (*p<0.05) was calculated using one way ANOVA. f, Schematic drawing of the treatment and imaging regiment for syngeneic mice implanted transthoracically with L3161C cell line and treated with Vehicle or Complex I inhibitor for the indicated time. g, Waterfall plot for % change in ¹⁸FBnTP uptake post-treatment to pre-treatment for mice treated with Vehicle (n=4 mice) or 500 mg/kg Metformin (n=5 mice) for 5 days. h, Waterfall plot for % change in ¹⁸FBnTP uptake post-treatment to pre-treatment for mice treated with Vehicle (n=4 mice) or 125 mg/kg Phenformin (n=6 mice) for 5 days. Experiments in e, g, and h were performed once. Statistical significance for g and h was calculated using unpaired two-tailed t-test.

FIG. 4 shows multi-tracer PET imaging of lung tumors in Kras/Lkb1 GEMMS with of ¹⁸FBnTP and ¹⁸F-FDG. a, Representative PET images of Kras/Lkb1 mice imaged with ¹⁸FBnTP and ¹⁸F-FDG on sequential days. Top panel shows CT and ¹⁸FBnTP image and bottom panel shows CT and ¹⁸F-FDG image. H—heart, T—tumor; tumors are indicated by arrows and circled. b, PET/CT images of a Kras/Lkb1 mouse with two tumors, T1 and T2. Top panel—¹⁸FBnTP/CT overlay, bottom panel—¹⁸F-FDG/CT overlay. H—heart. c, Western blot of tumors T1 and T2 isolated from mouse imaged in b and probed with indicated antibodies. d, Western blot from tumors isolated from Kras/Lkb1 (KL) or Kras/p53 (KP) mice was probed with indicated antibodies. e, Whole cell lysates from adenocarcinoma cell line A549 (ADC) and squamous cell carcinoma RH2 (SCC) cell lines were probed with indicated antibodies. Loading control (b-actin) is shown for each blot; intensity of bands relative to actin is indicated. f, TMRE measurement comparing A549 adenocarcinoma (ADC) (n=3) and RH2 squamous cell carcinoma (SCC) (n=3) human cell lines. g, Basal oxygen consumption rate (OCR) per cell was measured in ADC cells (A549) (n=15) (ADC) and SCC cells (RH2) (n=15). h, Extracellular acidification rate (ECAR) was measured in ADC cells (A549) (n=15) and SCC cells (RH2) (n=15). i, Coomassie staining of mitochondria isolated from ADC cells (A549) and SCC cells (RH2) separated on blue native gel. SC—supercomplex, I—complex I, V—complex V, III—complex III. Densitometry of the band corresponding to CI relative to band corresponding to CV and CIII is indicated. j, Complex I activity was evaluated by measuring oxygen consumption rate (OCR) per cell in ADC cells (A549) (n=25) and SCC cells (RH2) (n=25) when cells were using pyruvate and malate as substrates. k, Schematic drawing of the treatment regiment for Kras/Lkb1 mice. l, Percent of lung area occupied by tumor in Kras/Lkb1 mice treated according to k with Vehicle or 15 mg/kg IACS-010759. Each dot represents a mouse; n=9 (Vehicle), n=13 (IACS). m, Lung tumors from Kras/Lkb1 mice treated according to k were stained for proliferation marker Ki67 and percent positive cells per tumor area was quantified for TTF1 positive tumors (TTF1^{POS}) (left panel) and CK5 positive tumors (CK5^{POS}) tumors (right panel). n, Detailed view of a Kras/Lkb1 mouse imaged with ¹⁸FBnTP PET/CT (top panel) and ¹⁸F-FDG PET/CT (bottom panel). H—heart, tumor is outlined by dotted line. o, Tumor schematic of PET imaged tumor outlined in n with areas positive for ¹⁸FDG (red) and ¹⁸FBnTP (blue) color coded and labeled. p, The tumor shown in o was stained with Glut1 (left panel) and CK5/TTF1 (right panel). Tumor histology is indicated as ADC or SCC. The data are represented as the mean +/− SD. Statistical significance (*p<0.05; **p<0.01; ***p<0.001; ****p<0.0001; ns, not significant) was calculated using unpaired two-tailed t-test. Experiments in g, f, h, i, j were repeated twice.

FIG. 5 shows mitochondrial markers in Kras/Lkb1 mouse lung tumors. Whole cell lysates from lung tumors isolated from Kras/Lkb1 mice were immunoblotted with indicated antibodies. Tumors with high levels of surfactant protein C (SP-C:actin>0.5) are defined as adenocarcinomas (blue box), while tumors with lower levels of SP-C (SP-C:actin<0.5) are defined as squamous cell carcinomas (red box). The ratios of immunoblotted protein to actin are shown below each western blot panel.

FIG. 6 shows flow cytometry data from L3161C cells stained with TMRE. a, Gating strategy used for quantification of TMRE signal. The R2—region representing single cells was used for quantification of the TMRE signal. b, Overlay histogram showing shifts in TMRE signal in L3161C cells strained with Vehicle, 8 μM Oligomycin, and 8 μM Oligomycin+4 μM FCCP. c, Viability of A549 cells treated for 3 hr with vehicle or increasing doses of phenformin. d, Viability of L3161C cells treated for 3 hr with vehicle or oligomycin +/− FCCP. The data are represented as the mean +/− SD. Statistical significance (*p<0.05; **p<0.01; ***p<0.001; ****p<0.0001; ns, not significant) was calculated using one-way ANOVA. Experiments in c and d were repeated once.

FIG. 7 demonstrates that short-term treatment with phenformin does not induced changes in proliferation or apoptosis. a, Transverse ¹⁸FBnTP/CT overlay (left panel), image of the whole mouse lung after treatment with phenformin (middle panel), H&E stain of a lung lobe with associated adenocarcinoma (ADC) tumor (right panel). b, Representative slides stained with H&E (left panel), Cleaved Caspase 3 (middle panel), and Ki67 (right panel) from tumors from Kras/Lkb1 mice treated with Vehicle (top panel) or Phenformin (bottom panel). Scale bar=100 μm. c, Quantification of staining for Ki67 (left panel) and d, Cleaved Caspase 3 (right panel) for tumors from Kras/Lkb1 mice treated with Vehicle (n=5 mice) or Phenformin (n=5 mice). e, Phenformin was quantified using liquid chromatography/mass spectroscopy in lung tumors isolated from Kras/Lkb1 mice. Tumors were isolated from mice treated with Vehicle (saline) (n=6) or 100-200 mg/kg Phenformin (n=4) by oral gavage for 5 days. This experiment was repeated once. The data are represented as the mean +/− SD. Statistical significance (*p<0.05; **p<0.01; ***p<0.001; ****p<0.0001; ns, not significant) was calculated using unpaired two-tailed t-test.

FIG. 8 shows in vitro and in vivo analysis of the mouse lung adenocarcinoma cell line L3161C. a, H&E staining of a lung ADC from L3161C mouse tumor cell line. Scale bar=25 μm. b, Representative slides stained with Ki67 (top panel), and Cleaved Caspase 3 (bottom panel) from tumors formed by transthoracically transplanted L3161C cells that were treated with Vehicle, Metformin, Phenformin or Rotenone. Scale bar=200 μm. c, Basal OCR rate per cell for L3161C-pBabe (black) (n=6-12 replicates) and L3161C-ND1 cells (red) (n=12 replicates) treated with 50 μM Phenformin for 24 hr. d, Basal OCR rate per cell for L3161C-pBabe (black) (n=6-12 replicates) and L3161C-ND1 cells (red) (n=12 replicates) treated with indicated concentrations of Phenformin for 24 hr. This experiment was repeated once. The data are represented as the mean +/− SD. Statistical significance (***p<0.001; ****p<0.0001; ns, not significant) was calculated using one way ANOVA. e, Waterfall plot for % change in ¹⁸FBnTP uptake post-treatment to pre-treatment for mice transthoracically transplanted with L3161C cells expressing empty vector (L3161C+pBabe) (n=5 mice) or expressing ND1 (L3161C+ND1) (n=5 mice) and treated with 125 mg/kg/day Phenformin for 5 days. e, Waterfall plot of % change in ¹⁸FBnTP uptake in tumors formed by transthoracically transplanted L3161C-pBabe (n=3 mice) or L3161C-ND1 cells (n=6 mice) that were treated with Vehicle for 5 days. Experiments in e and f were preformed once. Statistical significance (*p<0.05; **p<0.01; ***p<0.001; ****p<0.0001; ns, not significant) was calculated using unpaired two-tailed t-test.

FIG. 9 shows IHC markers in lung tumors from Kras/Lkb1 mice. a, Whole lungs from Mouse 1, Mouse 2 and Mouse 3 stained for H&E or antibodies against Tom20, Glut1 or CK5-TTF1. Scale bar=5 mm. b, Representative images from tumor circled in red were stained with H&E or the indicated antibodies. Scale bar=25 μm.

FIG. 10 shows PET/CT and biochemical analysis of Kras/Lkb1 tumors. a-d, PET/CT images from Kras/Lkb1 mice that were imaged on sequential days with ¹⁸FBnTP (top panel) and ¹⁸F-FDG (bottom panel). Tumors are circled, H—heart. Maximum %ID/g uptake value for each tumor normalized to the maximum %ID/g uptake in the heart is indicated e, Western blot analysis from lung nodules that were isolated from mice imaged in a-d. Two lung tumors from mouse 5372 (imaged in b) are shown— T2 in red (high ¹⁸F-FDG and Glut1 levels; low ¹⁸FBnTP and low Ndufs1 and Ndufv1 levels); and T5 in blue (low ¹⁸F-FDG and Glut1 levels; high ¹⁸FBnTP and high Ndufs1 and Ndufv1 levels).

FIG. 11 shows a differential of Ndufs1 protein expression between ADC and SCC Kras/Lkb1 tumors. Whole cell lysates from lung tumors isolated from Kras/Lkb1 mice were immunoblotted with the indicated antibodies. The ratios of immunoblotted protein to actin are shown below each western blot panel.

FIG. 12 demonstrates the sensitivity of mouse and human lung cancer cell lines to complex I inhibitors phenformin and IACS-010759. a, TMRE measurement comparing mouse adenocarcinoma cell line (n=3) and mouse squamous cell carcinoma (n=3) cell lines. b, Cell viability of mouse adenocarcinoma cell line (n=3) and mouse squamous cell carcinoma cell line (n=3) was measured in the presence of indicated concentrations of Phenformin for 48 hr. c, Cell viability of human ADC cell line (A549) (n=3) and human SCC cell line (RH2) (n=3) was measured in the presence of indicated concentrations of Phenformin for 48 hr. d, Cell viability of human ADC cell line (A549) (n=3) and human SCC cell line (RH2) (n=3) was measured in the presence of indicated concentrations of IACS-010759 for 48 hr. The data are represented as the mean +/− SD. Statistical significance (*p<0.05; **p<0.01; ***p<0.001; ****p<0.0001; ns, not significant) was calculated using unpaired two-tailed t-test. Experiments were repeated twice.

FIG. 13 shows the characteristics of tumors from Kras/Lkb1 mice treated with Vehicle or IACS-010759. a, FBnTP uptake in tumors in Kras/Lkb1 mice before the start of the treatment with Vehicle or 15 mg/kg IACS-010759. Each dot represents a tumor; n=44 (Vehicle, 11 mice), n=68 (IACS, 15 mice). b and c, Images from H&E stained lung sections from Kras/Lkb1 mice treated with Vehicle (b) or 15 mg/kg IACS-010759 (c) for 12 days with tumors delineated with red lines. Quantification of this data is shown in FIG. 41. Scale bar=5 mm. The data are represented as the mean +/− SD. Statistical significance (*p<0.05; **p<0.01; ***p<0.001; ****p<0.0001; ns, not significant) was calculated using unpaired two-tailed t-test.

FIG. 14 shows IHC staining on a Kras/Lkb1 tumor with ADC-SCC mixed histology. Glut1 (left) and CK5-TTF1 (right) staining were performed on an ADC-SCC mixed tumor from a Kras/Lkb1 mouse. Tissue segmentation demarks ADC from SCC regions. Boxes indicated where representative 40× images were captured. Scale bar=1.0 mm.

FIG. 15 is a schematic view of an emission tomography system suitable for use with the present invention.

### DETAILED DESCRIPTION OF THE INVENTION

The present invention provides a method for detecting or ruling out non-small cell lung cancer (NSCLC) in a patient. The method includes the steps of: (a) administering to a patient a detectable amount of a compound of formula (I), wherein the compound is targeted to any NSCLC tumor in the patient; and (b) acquiring an image to detect the presence or absence of any NSCLC tumor in the patient.

Formula I is

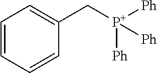 [SMILES: c1ccc(C[P+](c2ccccc2)(c2ccccc2)c2ccccc2)cc1]

In one version of the compound of formula (I), at least one of the atoms is replaced with a positron emitter. The positron emitter can be selected from the group consisting of ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

In one version of the compound of formula (I), preferably, the positron emitter is ¹⁸F.

Step (b) of the method includes acquiring the image using an imaging technique selected from positron emission tomography imaging, positron emission tomography with computed tomography imaging, or positron emission tomography with magnetic resonance imaging.

The magnetic resonance imaging contrast agent can be selected from the group consisting of ions of gadolinium, manganese, and iron. The metal ion can be paramagnetic.

Non-small cell lung cancer (NSCLC) is any type of epithelial lung cancer, other than small cell lung carcinoma (SCLC). NSCLC accounts for about 85% of all lung cancers. The most common types of NSCLC are squamous cell carcinoma, large cell carcinoma, and adenocarcinoma, but there are several other types that occur less frequently.

Mitochondria are required for lung tumorigenesis as was shown in a Kras^{G12D} driven genetically engineered mouse model (GEMM) of lung cancer. Mitochondria are essential for tumor initiation and maintenance as seminal experiments identified that loss of mtDNA inhibited mitochondrial bioenergetics and suppressed tumor cell growth in cell culture and xenografts.

A “detectable amount” means that the amount of the detectable compound that is administered is sufficient to enable detection of accumulation of the compound in a NSCLC cell or tumor by an imaging technique. A “patient” is a mammal, preferably a human, and most preferably a human suspected of having NSCLC.

The present invention also provides a method for evaluating mitochondrial complex I inhibition of (NSCLC) in a subject. The method includes the steps of: (a) administering an effective amount of a mitochondrial complex I inhibitor; (b) administering a detectable amount of a compound of formula (I); (c) waiting a time sufficient to allow the compound to accumulate at a tissue or cell site to be imaged; and (d) imaging the cells or tissues with an imaging technique.

Step (d) includes acquiring the image using an imaging technique selected from positron emission tomography imaging, positron emission tomography with computed tomography imaging, or positron emission tomography with magnetic resonance imaging.

A “mitochondrial complex I inhibitor” is compound capable of inhibiting mitochondrial complex I. Non-limiting examples of mitochondrial complex I inhibitors include metformin, phenformin, or rotenone.

At least one of the atoms in formula (I) used in this method is replaced with ¹⁸F.

A “subject” can also mean a “patient”.

The present invention also provides a method for evaluating mitochondrial complex V inhibition of a non-small cell lung cancer (NSCLC) in a subject. The method includes the steps of: (a) administering an effective amount of a mitochondrial complex V inhibitor; (b) administering a detectable amount of a compound of formula (I); (c) waiting a time sufficient to allow the compound to accumulate at a tissue or cell site to be imaged; and (d) imaging the cells or tissues with an imaging technique.

The imaging technique in step (d) is selected from positron emission tomography imaging, positron emission tomography with computed tomography imaging, or positron emission tomography with magnetic resonance imaging.

A “mitochondrial complex V inhibitor” is compound capable of inhibiting mitochondrial complex V. Non-limiting examples of mitochondrial complex I inhibitors include oligomycin.

At least one of the atoms in formula (I) used in this method is replaced with ¹⁸F.

The present invention also provides a method for evaluating mitochondrial complex II inhibition of a non-small cell lung cancer (NSCLC) in a subject. The method includes the steps of: (a) administering an effective amount of a mitochondrial complex II inhibitor; (b) administering a detectable amount of a compound of formula (I); (c) waiting a time sufficient to allow the compound to accumulate at a tissue or cell site to be imaged; and (d) imaging the cells or tissues with an imaging technique.

The imaging technique in step (d) is selected from positron emission tomography imaging, positron emission tomography with computed tomography imaging, or positron emission tomography with magnetic resonance imaging.

A “mitochondrial complex II inhibitor” is compound capable of inhibiting mitochondrial complex II.

The present invention also provides a method for evaluating mitochondrial membrane potential gradient (ΔΨ) in NSCLC in a subject. The method includes the steps of: (a) administering a detectable amount of a compound of formula (I) and (b) acquiring an image to detect the presence or absence of formula (I) in a NSCLC tumor in the subject.

The imaging technique in step (b) is selected from positron emission tomography imaging, positron emission tomography with computed tomography imaging, or positron emission tomography with magnetic resonance imaging.

At least one of the atoms in formula (I) used in this method is replaced with ¹⁸F.

A central process of the mitochondria is the synthesis of ATP through oxidative phosphorylation (OXPHOS) known as bioenergetics. The mitochondria maintain OXPHOS by creating a “membrane potential gradient (ΔΨ)” that is generated by the electron transport chain (ETC) in order to drive ATP synthesis. A “mitochondrial membrane potential gradient (ΔΨ)” can be measured via mass spectrometry. This invention provides the measurement of ΔΨ via PET imaging using a voltage sensitive compound.

The present invention also provides a method for evaluating mitochondrial membrane potential gradient (ΔΨ) in NSCLC in a subject. The method includes the steps of: (a) administering a detectable amount of a compound of formula (I); (b) acquiring an image to detect the presence or absence of formula (I) in a NSCLC tumor in the subject; (c) administering an effective amount of a mitochondrial complex I inhibitor; and (d) acquiring an image to detect the presence or absence of formula (I) in a NSCLC tumor in the subject.

The imaging technique in steps (b) and (d) is selected from positron emission tomography imaging, positron emission tomography with computed tomography imaging, or positron emission tomography with magnetic resonance imaging.

At least one of the atoms in formula (I) used in this method is replaced with ¹⁸F.

Non-limiting examples of mitochondrial complex I inhibitors include metformin, phenformin, or rotenone.

The present invention also provides a method evaluating mitochondrial complex I activity and mitochondrial membrane potential gradient (ΔΨ) in NSCLC in a subject. The method includes the steps of: (a) administering a detectable amount of a compound of formula (I) and (b) acquiring an image to detect the presence or absence of formula (I) in a NSCLC tumor in the subject.

The imaging technique in step (b) is selected from positron emission tomography imaging, positron emission tomography with computed tomography imaging, or positron emission tomography with magnetic resonance imaging.

At least one of the atoms in formula (I) used in this method is replaced with ¹⁸F.

The present invention also provides a method for detecting mitochondrial and metabolic heterogeneity within individual lung tumors in a subject. The method includes the steps of: (a) administering a detectable amount of a compound of formula (I); (b) administering a detectable amount of a compound of formula (II); and (c) acquiring an image to detect the presence or absence of formula (I) and formula (II) in a NSCLC tumor in the subject.

NSCLC is marked by genetic, metabolic and histological heterogeneity in tumors.

Formula (II) is

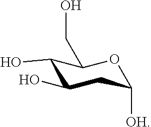

In one version of the compound of formula (II), at least one of the atoms is replaced with a positron emitter. The positron emitter can be selected from the group consisting of ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

In one version of the compound of formula (II), preferably, the positron emitter is ¹⁸F.

The imaging technique in step (b) is selected from positron emission tomography imaging, positron emission tomography with computed tomography imaging, or positron emission tomography with magnetic resonance imaging.

For the method above at least one of the atoms in formula (I) used in this method is replaced with ¹⁸F and at least one of the atoms in formula (II) used in this method is replaced with ¹⁸F.

The invention also provides a method for the treatment of lung adenocarcinoma (ADC) in a subject. The method includes the steps of: (a) administering a detectable amount of a compound of formula (I); (b) acquiring an image to detect the presence of an ADC tumor in the subject; and (c) administering an effective amount of a mitochondrial complex I inhibitor.

The imaging technique in step (b) is selected from positron emission tomography imaging, positron emission tomography with computed tomography imaging, or positron emission tomography with magnetic resonance imaging.

At least one of the atoms in formula (I) used in this method is replaced with ¹⁸F.

Non-limiting examples of mitochondrial complex I inhibitors include metformin, phenformin, IACS-010759, or rotenone.

An “effective amount” or “therapeutically effective amount” means an amount of a composition that, when administered to a subject for treating the condition, is sufficient to effect such treatment for the condition. The “effective amount” will vary depending on the composition, the severity of the condition treated, the age and relative health of the subject, the route and form of administration, the judgment of the attending medical or veterinary practitioner, and other factors. Those skilled in the art are readily able to determine effective amount by administering a compound until the condition is treated.

The invention also provides a method for the treatment of lung adenocarcinoma (ADC) in a subject. The method includes the steps of: (a) administering a detectable amount of a compound of formula (I); (b) acquiring an image to detect the presence of a Thyroid transcription factor 1 (TTF1)+ADC tumor in the subject; and (c) administering an effective amount of a mitochondrial complex I inhibitor.

The imaging technique in step (b) is selected from positron emission tomography imaging, positron emission tomography with computed tomography imaging, or positron emission tomography with magnetic resonance imaging.

At least one of the atoms in formula (I) used in this method is replaced with ¹⁸F.

Non-limiting examples of mitochondrial complex I inhibitors include metform in, phenform in, IACS-010759, or rotenone.

Lung adenocarcinoma (ADC) is a subtype of NSCLC. ADC starts in glandular cells, which secrete substances such as mucus, and tends to develop in smaller airways, such as alveoli. ADC is usually located more along the outer edges of the lungs. ADC tends to grow more slowly than other lung cancers.

The invention also provides for a method for evaluating electron transport chain (ETC) and oxidative phosphorylation (OXPHOS) of a non-small cell lung cancer (NSCLC) in a subject. The method includes (a) administering an effective amount of a small molecule; (b) administering a detectable amount of a compound of formula (I); (c) waiting a time sufficient to allow the compound to accumulate at a tissue or cell site to be imaged; and (d) imaging the cells or tissues with an imaging technique.

The imaging technique in step (d) is selected from positron emission tomography imaging, positron emission tomography with computed tomography imaging, or positron emission tomography with magnetic resonance imaging.

In some embodiments, the mitochondrial membrane potential increases after step (a).

A “small molecule” is a low molecular weight organic compound of less than 900 daltons (Da). In some embodiments, the small molecule is oligomycin.

In some embodiments, at least one of the atoms in formula (I) is replaced with ¹⁸F.

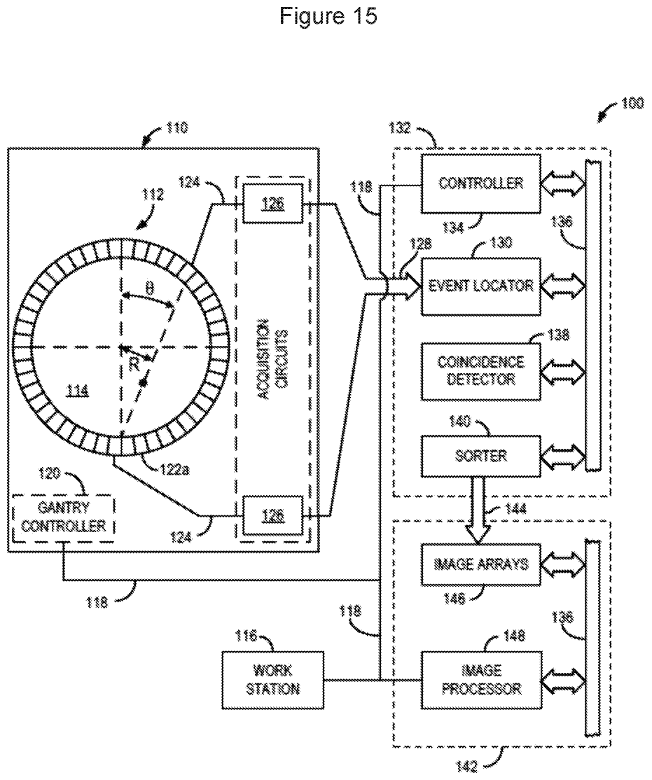

Referring to FIG. 15, a PET system 100 that can be used in the method of present invention includes an imaging hardware system 110 that includes a detector ring assembly 112 about a central axis, or bore 114. An operator work station 116 including a commercially-available processor running a commercially-available operating system communicates through a communications link 118 with a gantry controller 120 to control operation of the imaging hardware system 110.

The detector ring assembly 112 is formed of a multitude of radiation detector units 122 that produce a signal responsive to detection of a photon on communications line 124 when an event occurs. A set of acquisition circuits 126 receive the signals and produce signals indicating the event coordinates (x, y) and the total energy associated with the photons that caused the event. These signals are sent through a cable 128 to an event locator circuit 130. Each acquisition circuit 126 also produces an event detection pulse that indicates the exact moment the interaction took place. Other systems utilize sophisticated digital electronics that can also obtain this information regarding the precise instant in which the event occurred from the same signals used to obtain energy and event coordinates.

The event locator circuits 130 in some implementations, form part of a data acquisition processing system 132 that periodically samples the signals produced by the acquisition circuits 126. The data acquisition processing system 132 includes a general controller 134 that controls communications on a backplane bus 136 and on the general communications network 118. The event locator circuits 130 assemble the information regarding each valid event into a set of numbers that indicate precisely when the event took place and the position in which the event was detected. This event data packet is conveyed to a coincidence detector 138 that is also part of the data acquisition processing system 132.

The coincidence detector 138 accepts the event data packets from the event locator circuit 130 and determines if any two of them are in coincidence. Coincidence is determined by a number of factors. First, the time markers in each event data packet must be within a predetermined time window, for example, 0.5 nanoseconds or even down to picoseconds. Second, the locations indicated by the two event data packets must lie on a straight line that passes through the field of view in the scanner bore 114. Events that cannot be paired are discarded from consideration by the coincidence detector 138, but coincident event pairs are located and recorded as a coincidence data packet. These coincidence data packets are provided to a sorter 140. The function of the sorter in many traditional PET imaging systems is to receive the coincidence data packets and generate memory addresses from the coincidence data packets for the efficient storage of the coincidence data. In that context, the set of all projection rays that point in the same direction (θ) and pass through the scanner's field of view (FOV) is a complete projection, or “view”. The distance (R) between a particular projection ray and the center of the FOV locates that projection ray within the FOV. The sorter 140 counts all of the events that occur on a given projection ray (R, θ) during the scan by sorting out the coincidence data packets that indicate an event at the two detectors lying on this projection ray. The coincidence counts are organized, for example, as a set of two-dimensional arrays, one for each axial image plane, and each having as one of its dimensions the projection angle θ and the other dimension the distance R. This θ by R map of the measured events is call a histogram or, more commonly, a sinogram array. It is these sinograms that are processed to reconstruct images that indicate the number of events that took place at each image pixel location during the scan. The sorter 140 counts all events occurring along each projection ray (R, θ) and organizes them into an image data array.

The sorter 140 provides image datasets to an image processing/reconstruction system 142, for example, by way of a communications link 144 to be stored in an image array 146. The image arrays 146 hold the respective datasets for access by an image processor 148 that reconstructs images. The image processing/reconstruction system 142 may communicate with and/or be integrated with the work station 116 or other remote work stations.

The invention is further illustrated in the following Examples which are presented for purposes of illustration and not of limitation.

### EXAMPLES

Reference is made to the manuscript Momcilovic, Milica et al. “In vivo imaging of mitochondrial membrane potential in non-small-cell lung cancer.” Nature, vol. 575, 7782 (2019): 380-384, the content of which is incorporated by reference in its entirety.

The mitochondria are essential regulators of cellular energy and metabolism and they play a critical role in sustaining growth and survival of cancer cells. A central process of the mitochondria is the synthesis of ATP through oxidative phosphorylation (OXPHOS) known as bioenergetics. The mitochondria maintain OXPHOS by creating a membrane potential gradient (ΔΨ) that is generated by the electron transport chain (ETC) in order to drive ATP synthesis [Ref. 1,2]. Mitochondria are essential for tumor initiation and maintenance as seminal experiments identified that loss of mtDNA inhibited mitochondrial bioenergetics and suppressed tumor cell growth in cell culture and xenografts [Ref. 3,4,5]. However, our understanding of oxidative mitochondrial metabolism in cancer is limited because the majority of studies have been performed in vitro in cell culture models. This has left a large gap in our knowledge of how oxidative mitochondrial metabolism supports tumor growth and highlights a need for in vivo studies. Therefore we sought to measure mitochondrial ΔΨ in vivo in non-small cell lung cancer (NSCLC) using a voltage sensitive, positron emission tomography (PET) tracer known as 4-[18F]fluorobenzyl triphenylphosphonium (¹⁸FBnTP) [Ref. 6].

### METHODS AND MATERIALS

### Cell Culture

Cells were maintained at 3° C. in a humidified incubator with 5% CO₂. A549 cells were obtained from ATCC. The RH2 lung cancer cell line was previously established in our laboratories. All cell lines were routinely tested and confirmed to be free of Mycoplasma using the LookOut Mycoplasma PCR Detection Kit (Sigma). Cells were grown in Dulbecco's modified Eagle's medium (DMEM) plus 5% fetal bovine serum (Hyclone) and 1% penicillin/streptomycin (Gibco).

### Studies in GEMMS

We performed studies with the genetically engineered mouse model of lung cancer using Kras-Lox-Stop-Lox-G12D; Lkb1 Lox/Lox; Rosa26-Lox-Stop-Lox-Luc mice (KL) as described previously [Ref. 33]. Briefly, lung tumors were induced by intranasal administration of 5×10⁵ transduction units of Lenti-PGK-Cre (Cat. #FCT071, Kerafast,) as previously described [Ref. 9, 33]. Lentiviral delivery of Cre recombinase to KL mice leads to near 100% development of lung adenocarcinomas [Ref. 36]. KL mice that were inhaled with LentiCre were used in studies with Phenformin treatment. For studies where mice were imaged with both ¹⁸FBnTP and ¹⁸F-FDG probes, KL mice were inhaled with Adenoviral Cre, which leads to development of both ADC and SCC tumors. For all treatments, KL mice were imaged with ¹⁸FBnTP and sorted into two groups based on tumor maximum percent injected dose per gram (%ID/g) values, so that two groups would have similar maximum %ID/g values. Treatment was initiated on the same day or on the following day after ¹⁸FBnTP imaging. Mice were treated with 125 mg/kg/day phenformin for 5 days or 15 mg/kg/day IACS-010759 for 12 days. The drugs were delivered by oral gavage. All experimental procedures that were performed on mice were approved by the UCLA Animal Research Committee (ARC). Both male and female mice were used in all experiments and no preference in mouse gender was given for any of the studies.

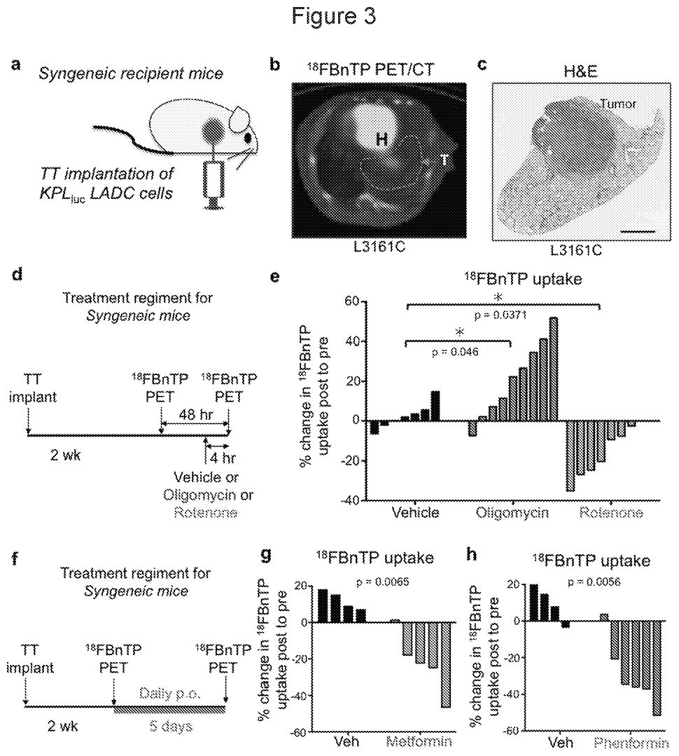

¹⁸FBnTP tracer can be used to detect increases in the mitochondrial membrane potential. Increases in the mitochondrial membrane potential have been connected to the chemotherapy resistance (Montero et al 2015 PMID 25723171) and tumor progression. However, increases in mitochondrial membrane potential were detected in vitro, greatly limiting clinical utility. We show that ¹⁸FBnTP tracer can detect increases in mitochondrial membrane potential in vivo (FIG. 3). We implanted KPL adenocarcinoma cell line into syngeneic mice, and imaged mice with ¹⁸FBnTP to detect baseline uptake of the tracer. Next we treated mice with a single dose of vehicle, oligomycin (complex V inhibitor), rotenone (complex I inhibitor) and imaged mice with ¹⁸FBnTP 3-4 hr after treatment (FIGS. 3a and d). We detected increases in ¹⁸FBnTP upon acute treatment with oligomycin (complex V inhibitor) (FIG. 3e), thus validating ¹⁸FBnTP as a probe that can detect increases in mitochondrial membrane potential in response to drugs that interfere with the function of proteins that contribute to the maintenance of the mitochondrial membrane potential.

### Studies in Syngeneic Mice

We established the L3161C mouse cell line from a lung tumor dissected from a Kras;Lkb1;p53 (KPL) mouse. Following resection, the tumor was minced and incubated in collagenase/dispase (Cat. #10269638001, Sigma) for 3 hr. Cells were filtered using a 70 μm strainer, centrifuged, resuspended in DMEM with 5% FBS and plated in a 6 cm dish. The following day media was changed and cells that attached were cultured. We confirmed that the L3161C cell line was adenocarcinoma by implanting cells in syngeneic mice, detecting lung tumors and staining tumor with H&E and CK5/TTF1. For imaging studies, 1x10⁵ L3161C cells suspended in 20 μl PBS were implanted into the left lung lobe via transthoracic injection [Ref. 37]. Two weeks after injections, mice were imaged by CT. Mice with similar sized tumors were used for ¹⁸FBnTP imaging. For treatment studies, syngeneic mice were imaged with ¹⁸FBnTP, and split into two groups (three groups for FIG. 3e) based on tumor maximum %ID/g, such that maximum %ID/g of tumors in both groups would be similar. Treatment was then initiated for the specified time. Mice were treated with a single dose of 0.25 mg/kg Oligomycin or 0.5 mg/kg Rotenone; both drugs were delivered by i.p. injection. For other studies mice were treated with 125 mg/kg/day phenformin or 500 mg/kg/day metformin for 5 days; both drugs were delivered by oral gavage in the morning. Following imaging, mice were euthanized and tissue was harvested. For some experiments, lungs were fixed with 10% NBF overnight; for other experiments lung tumor nodules were rapidly dissected, snap frozen in liquid nitrogen and stored in −80° C. freezer.

### ¹⁸FBnTP Synthesis

We performed synthesis of the radio-tracer ¹⁸FBnTP as previously described [Ref. 16]. The three-pot, four-step synthesis of [¹⁸F]FBnTP was performed using the automated radiochemical synthesizer ELIXYS FLEX/CHEM (Sofie Biosciences). The no-carrier-added [¹⁸F]fluoride was produced from the (p,n) reaction of [¹⁸O]H₂O with an RDS-112 11 MeV cyclotron (Siemens) in a 1-ml tantalum target with Havar foil. [¹⁸F]Fluoride in water was pushed through a strong cation exchange (SCX) cartridge and trapped on a QMA cartridge. [¹⁸F]Fluoride was then eluted with a solution of Kryptofix 222 (10 mg, 27 μmol) and K₂CO₃ (1 mg, 7 μmol) in an acetonitrile/water (3:5, 0.8 ml) mixture. Azeotropic evaporation was performed at 110° C. under a stream of nitrogen (7 psi) to remove excess water using acetonitrile. The 4-trimethylammoniumbenzaldehyde trifluoromethansulfonate (5 mg) precursor was solvated in DMSO (0.8 ml), added to the reactor vial containing the dried [¹⁸F]fluoride and allowed to react at 90° C. for 5 min with stirring. The resulting 4-[¹⁸F]fluorobenzaldehyde mixture was diluted with water containing 1% (w/v) Na-ascorbate solution (5 ml total) and passed through an Oasis WCX cartridge (6 psi) for 1.5 min. The WCX cartridge was dried with nitrogen (20 psi) for 1 min and eluted with DCM (3 ml). The mixture was passed through a glass column containing NaBH₄.(Al₂O₃)ₓ (350 mg) on the top half portion and K₂CO₃ (2 g) on the bottom half portion for a flow through reduction of 4-[¹⁸F]fluorobenzaldehyde ([¹⁸F]FBA) to 4-[¹⁸F]fluorobenzyl alcohol ([¹⁸F]FBnOH), which was directed to the second reactor vial (3 psi). A subsequent elution and rinsing of the column was performed using DCM (1 ml, containing 0.2% (v/v) of water) (3 psi). The mixture containing [¹⁸F]FBnOH was reacted with Ph₃PBr₂ (100 mg) in DCM (1.1 ml) at 35° C. for 10 min resulting in the formation of 4-[¹⁸F]fluorobenzyl bromide ([¹⁸F]FBnBr). The resulting mixture was passed through a silica cartridge and directed towards the third reactor vial (2 psi). A solution of PPh₃ (3 mg) in EtOH (0.6 ml) was added, followed by removal of most of the DCM under vacuum and a stream of nitrogen (3 psi) at 45° C. for 6.5 min while stirring. EtOH (1 ml) was added and the mixture was evaporated to approximately 0.5 ml under vacuum and a stream of nitrogen (7 psi) at 80° C. for 2.5 min while stirring. The mixture was reacted at 160° C. for 5 min in a sealed position, which converted the [¹⁸F]FBnBr to the desired [¹⁸F]FBnTP. The reaction vial was cooled to 35° C. and diluted with water (3 ml) while stirring. The mixture was passed through a Sep-Pak Plus Accell CM cartridge (8 psi) and the cartridge was washed with EtOH (20 ml). The product was released with 2% EtOH in saline +0.5% (w/v) Na-ascorbate (10 ml) and passed through a sterile filter into a vented sterile vial. Under optimized conditions, the resulting [¹⁸F]FBnTP PET tracer was obtained in AY of 1.4-2.2 GBq starting from 9.4 to 12.0 GBq [¹⁸F]fluoride in 90-92 min (RCY=28.6±5.1% with n=3). Molar activities ranged from 80 to 99 GBq/μmol (EOS) and radiochemical purity was >99%.

### PET/CT Imaging Studies

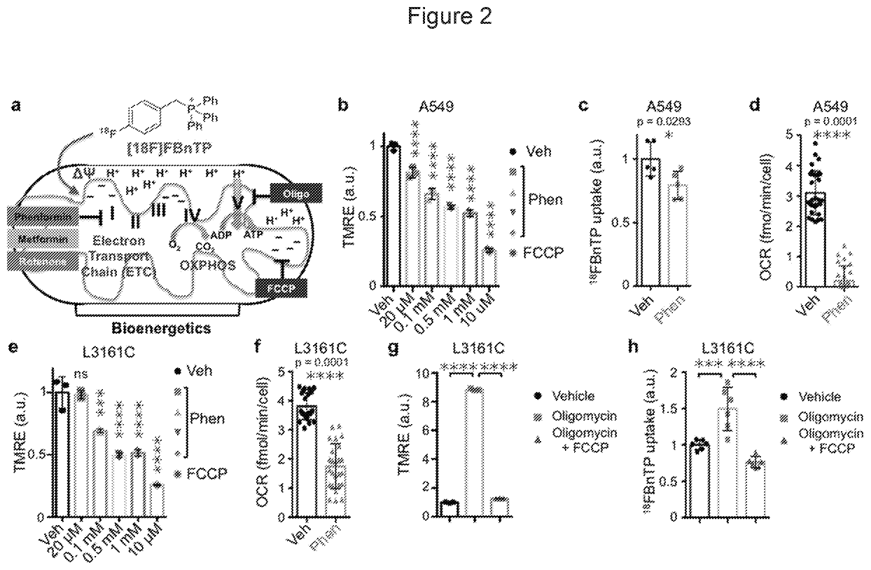

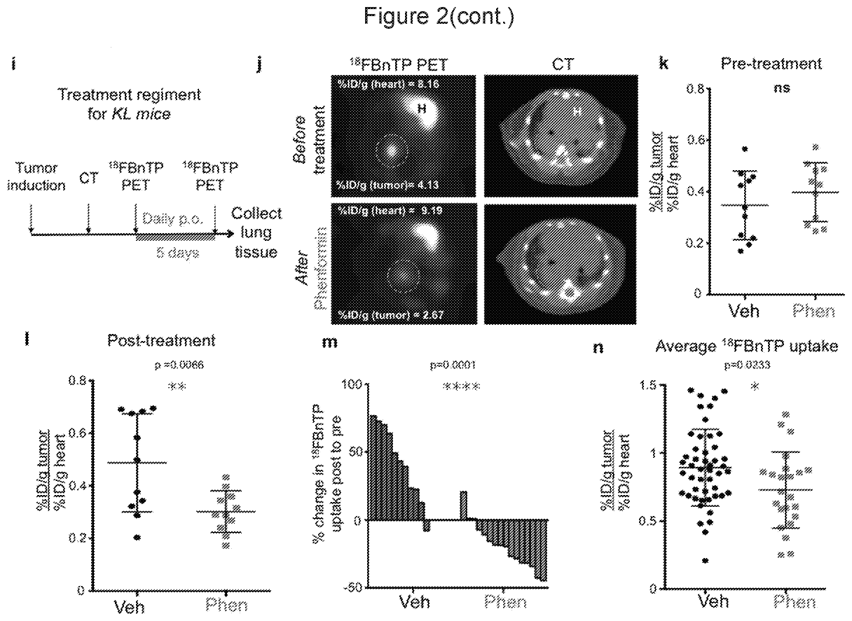

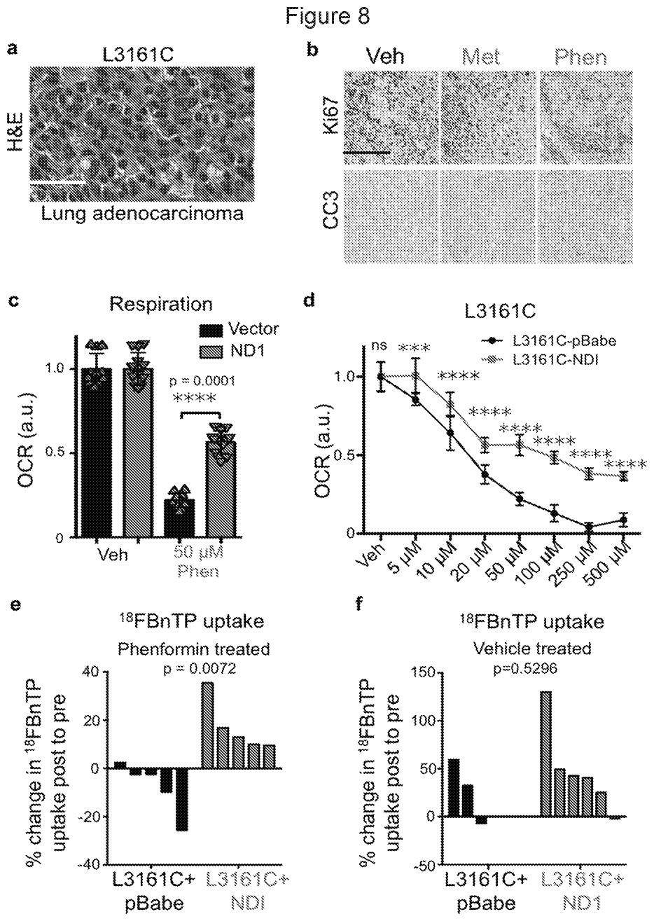

All PET/CT imaging was performed on KL mice or syngeneic mice TT implanted with L3161C using either single tracer imaging with ¹⁸FBnTP or dual radio-tracer imaging with ¹⁸FBnTP and ¹⁸F-FDG as previously described [Ref. 33,38]. In order to reduce variability between mice imaged with ¹⁸FBnTP, maximum percent injected dose per gram (%ID/g) for each tumor was normalized to maximum %ID/g of heart as indicated in the figures. For waterfall plots in FIG. 2m and FIGS. 8e-8f, we calculated % change in the uptake of the ¹⁸FBnTP probe post-treatment relative to pre-treatment using maximum %ID/g of the tumor that was normalized to maximum %ID/g of the heart for each mouse.

### Respirometry Analysis

Experiments were conducted on a Seahorse XF96 Extracellular Flux Analyzer (Agilent Technologies) in order to measure oxygen consumption rate (OCR) and extracellular acidification rate (ECAR). L3161C, A549 or RH2 cells were seeded into an XF96 microplate at density of 12,000-15,000 cells per well. Cells were plated in growth medium and maintained overnight in a tissue culture incubator (37° C.; 5% CO₂). On the day of the experiment, assay medium (Seahorse XF Base Medium supplemented with 2 mM L-glutamine, 1mM Pyruvate and 10 mM glucose) was freshly prepared. The cells were washed twice with assay medium and brought to a final volume of 175 μL per well. The XF96 plate was placed in a 37° C. incubator without CO₂ for 30 minutes prior to loading the plate into the instrument. Injection of compounds during the assay included: the mitochondrial ATP Synthase inhibitor oligomycin (final concentration of 2 μM); the chemical uncoupler, FCCP (final concentration of 1 μM); and the Complex I inhibitors rotenone (final concentrations 2 μM) and phenformin (final concentration 1 mM) and Complex III inhibitor antimycin A (final concentrations of 2 μM). At the conclusion of the assay, the cells were fixed with 4% paraformaldehyde, stained with Hoechst, and cell number per well was determine based on nuclei number using an Operetta High-Content Imaging System (PerkinElmer). Oxygen consumption rates were normalized to cell number per well. Activity of complex I was measured in permeabilized cells using XF PMP assay where complex I dependent OCR was measured by determining OCR in the presence of pyruvate and malate (as substrates for complex I) before and after addition of rotenone (complex I inhibitor).

### Measurement of Mitochondrial Membrane Potential (ΔΨ)

Experiments were conducted by analyzing TMRE fluorescence using flow cytometry. A549 and L3161C cells were harvested on the day of the assay and 500,000 cells were aliquoted into a microfuge tube. Cells were resuspended in 0.5 ml DMEM with 5% FBS containing different concentrations of phenformin or oligomycin or FCCP as indicated in figure legends. Cells were incubated in a tissue culture incubator (37° C., 5% CO₂) for 2 hr. After 2 hr incubation, media with TMRE was added to tubes, such that final concentration of TMRE was 7 nM and concentrations of phenformin, oligomycin and FCCP were constant. Cells were incubated for an additional 1 hr in a tissue culture incubator (37° C., 5% CO₂). Cells were washed twice in phenol red free DMEM and cellular fluorescence was acquired with a BD LSRII analyzer at UCLA Flow Cytometry Core. Data was analyzed with Flowing Software.

### In Vitro ¹⁸FBnTP Uptake Assay

On the day of the assay, cells were trypsinized, collected in DMEM with 5% FBS and counted. 1,000,000 cells were aliquoted into a microfuge tube, resuspended in 0.5 ml media containing DMEM with 5% FBS and either Vehicle, 1 mM phenformin, 8 μM Oligomycin or 8 μM Oligomycin with 4 μM FCCP. Cells were incubated in a tissue culture incubator (37° C., 5% CO₂) for 2 hr. After 2 hr incubation, 0.5 ml of media with ¹⁸FBnTP was added to tubes, such that final concentration of ¹⁸FBnTP was 10 μCi/ml and concentrations of phenformin, oligomycin and FCCP were constant. Cells were incubated for an additional 1 hr in a tissue culture incubator (37° C., 5% CO₂). The uptake was terminated by centrifugation at 4° C. (1200 rpm, 5 min). Cells were washed twice with cold media. After the final wash, cell pellet was resuspended in 500 μl of media, and 300 μl was used in a gamma counter, while 100 μl was used to count viable cells using ViCell counter (Beckman). Counts per minute were normalized to viable cells.

### Immunohistochemistry

After the fixation step in 10% neutral buffered formalin overnight, lungs were transferred to 70% ethanol and further processing and embedding was done by the TPCL at UCLA. The following antibodies were used: anti-CK5 (EP1601Y) (abcam, ab52635 1:100), anti-TTF1 (8G7G3/1) (Dako, 1:1000), anti-Ki67 (SP6) (Thermo-Scientific, RM-9106-SO 1:200), anti-Glut1 (alpha-diagnostic, GT11-A, 1:400). Slides were scanned onto a ScanScope AT (Aperio Technologies Inc.). Digital slides were analyzed with Definiens and QuPath software.

### Western Blot Analysis

Whole cell lysates from lung tumors isolated from mice were prepared as previously described [Ref. 9]. Briefly, tumors were homogenized in buffer containing phosphatase and protease inhibitors (20 mM Tris pH 7.5, 150 mM NaCI, 1% Triton X-100, 50 mM sodium fluoride, 1 mM EDTA, 1 mM EGTA, 2.5 mM pyrophosphate, 1 mM sodium orthovanadate, protease inhibitor tablet), centrifuged and supernatant was normalized, aliquoted and stored in -80° C. freezer. Lysates were separated on 4-12% Bis-Tris protein gels (Thermo), transferred to PVDF membrane and probed with the following antibodies: SP-C (1:5000, AB3786 Milipore); Glut1 (1:2000, GT11-A, Alpha Diagnostic); Ndufs1 (1:1000, ab169540, abcam); Ndufs1 (1:1000, sc-271510, Santa Cruz); Ndusvl (1:1000, 11283-1-AP, Proteintech), Tom20 (1:10000, FL-145, Santa Cruz), Tom40 (1:2000, 18409-1-AP, Proteintech); Tom70 (1:2000, 14528-1-AP, Proteintech); Tim23 (1:2000, 11123-1-AP, Proteintech); actin (1:5000, 4967, Cell Signaling Technology). Intensity of bands was quantified using Image J.

### Blue Native Gel

Blue Native (BN)-PAGE was performed based on the method of Schagger and colleagues [Ref. 39] with minor modifications. Briefly, mitochondria (100 μg protein) were solubilized for 15 min with digitonin using a 6g/g digitonin/protein ratio. Insoluble material was removed by centrifugation at 21,000 g for 30 min at 4° C., the soluble component was combined with BN-PAGE loading dye and separated on a 3-13% acrylamide-bisacrylamide precast BN-PAGE gel. For separation, cathode buffer (15 mM bis-Tris [pH 7.0] and 50 mM tricine) containing 0.02% (w/v) Coomassie Blue G was used until the dye front had reached approximately one-third of the way through the gel before exchange with cathode buffer lacking Coomassie Blue G. Anode buffer contained 50 mM bis-Tris (pH 7.0). Native complexes were separated at 4° C. at 110V for 1 hour, followed by 12mA constant current. Thyroglobulin (669 kDa), ferritin (440 kDa), Catalase (232 kDa), Lactate dehydrogenase (140 kDa), and bovine serum albumin (BSA 67 kDa) were used as markers (GE Healthcare).

### Liquid Chromatography/Mass Spectroscopy

Tumors were homogenized with a Tissue Master (Omni international) in 1 ml chilled 80% Methanol. Tumor suspensions were spun down at 4° C. for 5 min at 17,000g, and the top layer taken as extracted metabolites. The volume equivalent of 1 mg of tumor was transferred into glass vials and the samples were dried with a EZ2-Elite lyophilizer (Genevac). Dried metabolites were re-suspended in 100 μl of 50%:50% acetonitrile (ACN):dH20 solution; 10 μl of these suspensions were injected per analysis. Samples were run on a Vanquish (Thermo Scientific) UHPLC system with mobile phase A (5 mM NH4AcO, pH 9.9) and mobile phase B (ACN). Separation was achieved at a 200 μl/min flow rate on a Luna 3mm NH2 100A (150×2.0 mm) at 40° C. with a gradient going from 15% A to 95% A in 18 min followed by an 11 minute isocratic step. The UHPLC was coupled to a Q-Exactive (Thermo Scientific) mass analyzer running in positive mode at 3.5 kV with an MS1 resolution of 70,000. Metabolites were identified using exact mass (MS1), retention time, and fragmentation patterns (MS2) at normalized collision energy (NCE) 35. Quantification was performed via area under the curve (AUC) integration of MS1 ion chromatograms with the MZmine 2 software package. For the quantification of absolute moles of phenformin, one tumor from the vehicle group was selected to provide a representative tumor small molecular matrix. The volume equivalent of 1 mg of this tumor was distributed into several glass vials and 10 μl of pure aqueous phenformin standards (0.1mM - 0.5mM) was added to these samples to span the possible range of phenformin concentrations. From this point on these samples were treated as described above. AUC values from the phenformin standards were used to fit a linear regression model that related MS1 AUC to the moles of phenformin present. The linear regression equation was used to convert MS1 AUC to moles of phenformin in all tumor samples and expressed relative to the tissue mass of each tumor.

### Statistical Analysis

The in vivo experiments were analyzed utilizing analysis of variance (ANOVA) models to evaluate the main effects of the two treatment types on the various quantitative outcome measures. Categorical outcomes were compared between groups with Fisher's exact test. The sample size of 7-10 mice per group provided a 99% power to detect differences in the outcomes of % cleaved caspase 3, Ki67 and ¹⁸FBnTP %ID/g positivity based on the observed results previously described [Ref. 9], assuming a two-sample t-test (a simplification of the ANOVA analysis plan) with a two-sided 0.05 significance level.

Mouse experiments involving imaging of Kras/Lkb1 mice with ¹⁸FBnTP were repeated with 3 separate cohorts several months apart. For imaging studies with both GEMMS and syngeneic mice, after basal ¹⁸FBnTP imaging, mice were split into two groups (3 cohorts for FIG. 3e) based on maximum %ID/g values, such that maximum %ID/g values of tumors in both groups would be similar. For all experiments, investigators were not blinded to the group allocation during treatment or during analysis. Variation is indicated using standard deviation or standard error of the mean as described in the figure legends. Differences between groups were determined using unpaired two-tailed t-test or one-way ANOVA if more than two groups were compared. Western blot analysis of mitochondrial markers was completed on lung nodules isolated from four separate cohorts of mice.

### Results and Discussion

We used ¹⁸FBnTP PET imaging to profile mitochondrial ΔΨ in autochthonous mouse models of lung cancer and discovered distinct functional mitochondrial heterogeneity within NSCLC tumor subtypes. The use of ¹⁸FBnTP PET imaging enabled us to functionally profile mitochondrial ΔΨ in live tumors. We anticipate ¹⁸FBnTP PET imaging will have dynamic applications beyond that of cancer and benefit a broad range of fields focused on understanding how mitochondria impact human disease.

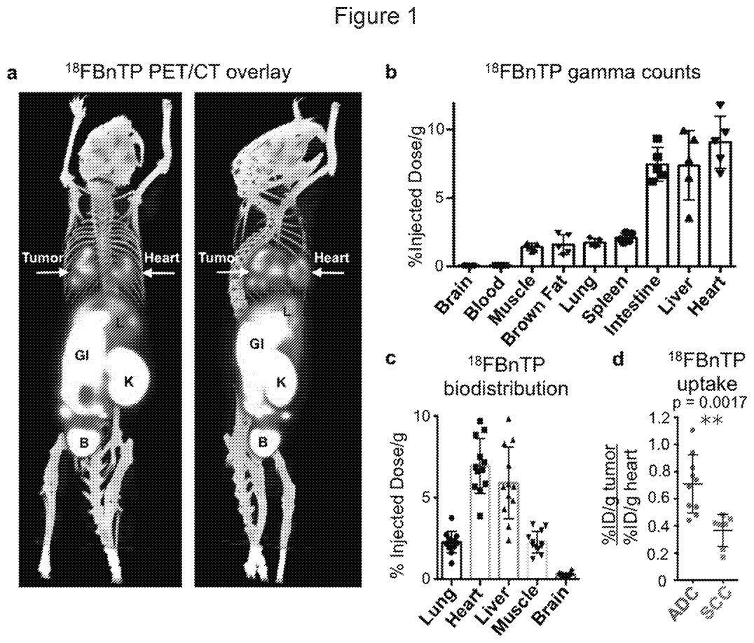

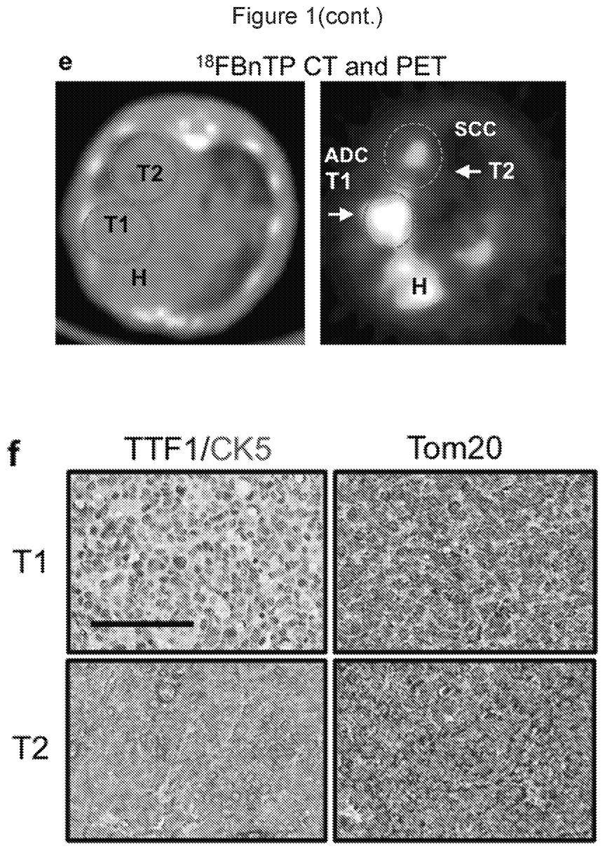

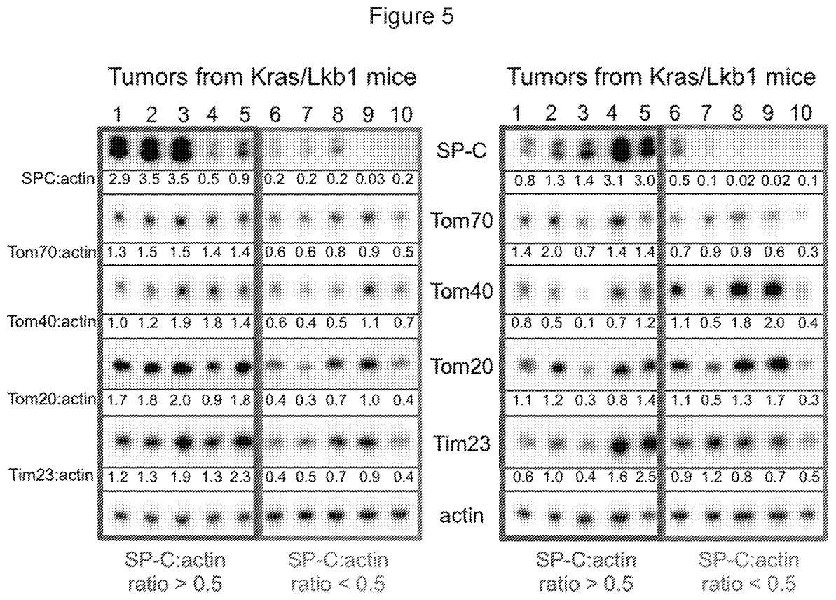

Mitochondria are required for lung tumorigenesis as was shown in a Kras^{G12D} driven genetically engineered mouse model (GEMM) of lung cancer [Ref. 7]. Therefore we used Kras^{G12D} mutant, Lkb1 deficient (Kras/Lkb1) GEMMS to perform ¹⁸FBnTP PET imaging on lung tumors in vivo [Ref. 8,9]. ¹⁸FBnTP is a positively charged lipophilic cation that localizes to the negatively charged mitochondrial inner membrane in a voltage-dependent manner [Ref. 10,11]. The probe has most frequently been studied in rodent and canine myocardium to detect myocardial infarction [Ref. 6,12] and in cancer cell xenografts as a surrogate marker of apoptosis following cytotoxic treatment with chemotherapy agents [Ref. 13,14]. While mass spectroscopy based approaches have been used to study ΔΨ [Ref. 15], no study to date has used ¹⁸FBnTP PET to measure mitochondrial ΔΨ using autochthonous murine models of lung cancer. We first synthesized ¹⁸FBnTP as previously described [Ref. 16] and performed PET imaging on lung tumors in Kras/Lkb1 mice ten weeks post tumor induction. We identified both ¹⁸FBnTP positive lung tumors and heart (FIG. 1a). We then performed biodistribution analysis of tissues by either measuring gamma counts or percent injected dose per gram (%ID/g) and confirmed high uptake of the tracer in the heart, liver and intestine as well as low uptake in normal lung, skeletal muscle and brain (FIGS. 1b,c). Analysis of ¹⁸FBnTP PET imaged Kras/Lkb1 mice identified two distinct populations of lung tumors distinguished by either high or low ¹⁸FBnTP uptake (FIG. 1d,e). Interestingly, we confirmed that tumors with high ¹⁸FBnTP avidity segregated with lung adenocarcinomas (ADCs) while lung squamous cell carcinomas (SCC) tumors had uniformly lower avidity for ¹⁸FBnTP (FIG. 1d). We confirmed lung tumor histology by staining tumors for cytokeratin 5 (CK5) to mark SCC and thyroid transcription factor 1 (TTF1) or surfactant protein C (SP-C) to identify ADCs (FIG. 1f; FIG. 5). We suspected that lung SCC with low ¹⁸FBnTP uptake had low mitochondrial content thus explaining the reduced ΔΨ and ¹⁸FBnTP uptake. We stained tumors for the pan mitochondrial marker Tom20 and confirmed similar staining intensities for both lung ADC and SCC (FIG. 1f). We performed additional analysis of the mitochondrial membrane proteins Tom20, 40, 70 and Tim23 in lung ADC and SCC from KL mice and showed that ADCs (SP-C:actin ratio>0.5) had no discernable difference in expression of these proteins as compared to SCC (SP-C:actin ratio<0.5) (FIG. 5). These results demonstrate that both tumor subtypes have similar mitochondrial content but a two-fold difference in ¹⁸FBnTP affinity (FIG. 1d).

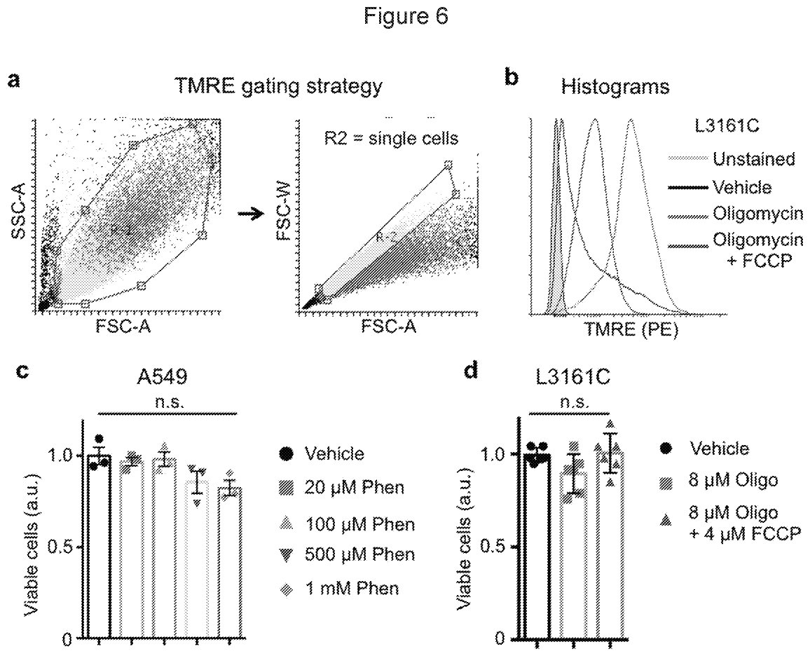

¹⁸FBnTP functions as a surrogate marker of mitochondrial ΔΨ in vivo [Ref. 17], therefore we sought to validate ¹⁸FBnTP as a voltage sensitive marker of both ΔΨ and OXPHOS by treating cells with mitochondrial complex I inhibitor phenformin, which dissipates ΔΨ and inhibits OXPHOS [Ref. 18] (FIG. 2a). Short-term phenformin treatment of the human lung ADC cell line A549 or the mouse lung ADC line L3161C (derived from a Kras^{G12D};p53^{-/-};Lkb1^{-/-} mouse) significantly reduced ΔΨ in a dose dependent manner as measured by TMRE staining (FIGS. 2b, 2e). The flow cytometry gating parameters for TMRE analysis and cell viability following acute phenformin treatment are represented in Extended Data FIG. 2a-c. Likewise, acute treatment of A549 cells with phenformin significantly reduced ¹⁸FBnTP uptake similar to results with TMRE staining (FIG. 2c). We next demonstrated that loss of ΔΨ induced by phenformin also resulted in a significant reduction of cellular OXPHOS in both A549 and L3161C cells as measured by respirometry (FIG. 2d, 2f). Lastly, we treated L3161C cells with oligomycin, a complex V ATPase inhibitor, which induces increased ΔΨ. We detected a significant increase in TMRE and ¹⁸FBnTP uptake following oligomycin treatment while ΔΨ was significantly reduced following the addition of the mitochondrial uncoupler FCCP (FIGS. 2g, h; FIG. 6b) matching a response equal to the in vivo probe MitoClick [Ref. 15]. Acute treatment with oligomycin +/− FCCP did not affect cell viability (FIG. 6d). These results demonstrate that ¹⁸FBnTP is a voltage sensitive probe that detected changes in ΔΨ and OXPHOS in mouse and human lung cancer cells.

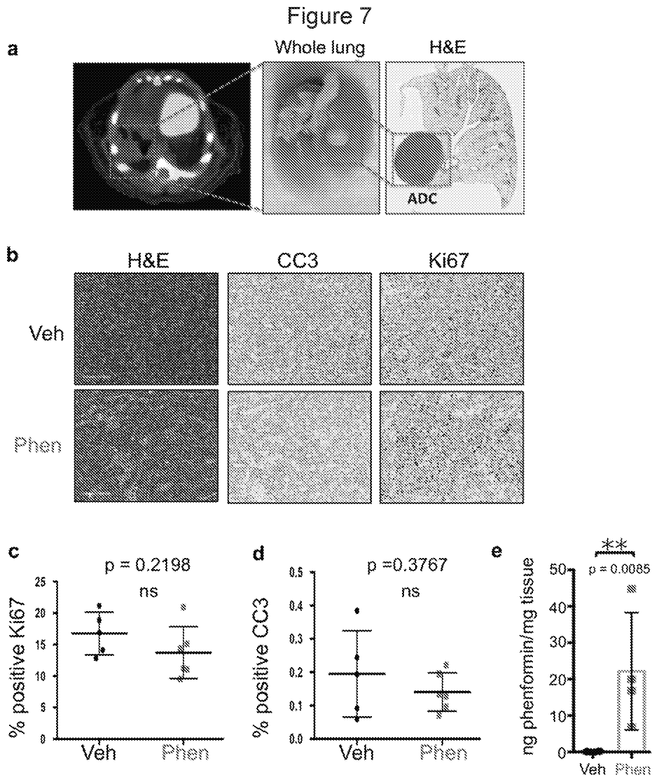

We next measured ¹⁸FBnTP uptake in lung tumors of Kras/Lkb1 GEMMS before and after treatment with either phenformin or vehicle (saline) as described in FIG. 2i. Representative PET images show reduced ¹⁸FBnTP uptake in lung tumors following phenformin treatment (FIG. 2j). Treatment groups showed no significant difference in ¹⁸FBnTP uptake values before start of the treatment (FIG. 2k) but showed a significant reduction in ¹⁸FBnTP uptake in the mice that received phenformin (FIG. 2l). We further quantified the fold change in ¹⁸FBnTP uptake following treatment and show that phenformin induced a significant drop in tracer uptake compared to vehicle treated mice (FIG. 2m). We performed three independent experiments in Kras/Lkb1 mice that showed a significant reduction in ¹⁸FBnTP uptake in phenformin treated lung tumors compared to those treated with vehicle alone (FIG. 2n). Because Kras/Lkb1 tumors are sensitive to phenformin [Ref. 9] it is possible that reduced ¹⁸FBnTP uptake was due to phenformin induced cell death. Therefore, we stained phenformin and vehicle treated tumors for cleaved caspase 3 and Ki67 and found no evidence of phenformin induced apoptosis or reduced cell viability (FIGS. 7a-d). This data agrees with previously published studies in which only a longer duration of phenformin treatment between 4-8 weeks resulted in cellular apoptosis [Ref. 9,19]. Measurement of phenformin by mass spectrometry in lung tumor tissue from KL mice showed a significant increase in phenformin in tumors from treated mice as compared to tumors treated with vehicle (FIG. 7e).

We then investigated whether ¹⁸FBnTP could selectively measure changes in ΔΨ and OXPHOS in vivo following treatment with a broader panel of ETC inhibitors. The complex I inhibitors included metformin, phenformin, or rotenone, which all reduce ΔΨ and suppress OXPHOS [Ref. 9,18,20] as well as the complex V inhibitor oligomycin, which induces an increase in ΔΨ. In order to accurately and rapidly profile ΔΨ in lung tumors using ¹⁸FBnTP PET, we employed an orthotopic mouse model in which we transthoracically (TT) implanted L3161C lung tumor cells into the left lobe of syngeneic recipient mice shown in FIG. 3a. Importantly, this enabled us to spatially deliver a single lung tumor in the left lobe of the mouse and perform ¹⁸FBnTP PET imaging. L3161C lung ADC tumor cells retained their mitochondrial ΔΨ and ¹⁸FBnTP avidity following TT implantation into syngeneic mice shown in FIG. 3b. Hematoxylin and eosin (H&E) staining of the left lung lobe confirmed that L3161C cells formed well-differentiated lung ADCs (FIG. 3c; FIG. 8a).

We next measured ¹⁸FBnTP uptake in syngeneic mice TT implanted with L3161C cells following acute 4 hour treatment with either oligomycin or rotenone [Ref. 21] (FIG. 3d). We used a safely tolerated dose of rotenone (0.5mg/kg/day) or oligomycin (0.25mg/kg) below the toxic dose range [Ref. 22]. We show that ¹⁸FBnTP uptake in lung tumors was significantly increased after delivery of oligomycin whereas rotenone treatment significantly reduced ¹⁸FBnTP uptake (FIG. 3e). Our results demonstrate that ¹⁸FBnTP PET detected acute changes in ΔΨ in lung tumors following inhibition of complex I or V, agreeing with in vitro experiments described in FIG. 2.

At a cellular level metformin, like phenformin, inhibits mitochondrial complex I resulting in reduced OXPHOS [Ref. 23-27], and is broadly used worldwide to clinically manage type 2 diabetes [Ref. 28]. Yet, despite decades of clinical use and research on metformin there has been no definitive biomarker established to measure its direct inhibition of complex I activity in vivo. We therefore sought to determine if ¹⁸FBnTP PET could measure changes in the ΔΨ of lung tumors following systemic treatment of mice with metformin (FIG. 3f). Our results show that ¹⁸FBnTP uptake in lung tumors was significantly reduced in the mice that received metformin compared to the vehicle treated mice (FIG. 3g). Metformin is a less potent inhibitor of complex I than phenformin and as expected it had slightly less inhibition of ¹⁸FBnTP uptake than did phenformin (FIGS. 3g and 3h). Histological analysis of tumors treated with biguanides confirmed that neither metformin nor phenformin induced apoptosis, cell death or significantly altered tumor growth measured by CC3 and Ki67, respectively (FIG. 8b). These results confirm that ¹⁸FBnTP imaging can accurately detect a loss of ΔΨ following delivery of metformin.

We next sought to perform a rescue experiment using the Saccharomyces cerevisiae NADH dehydrogenase (ND1), which oxidizes NADH similar to mammalian mitochondrial complex I. Expression of the yeast ND1 protein enables cells to bypass complex I inhibition rendering them insensitive to metformin or phenformin [Ref. 26,29]. Expression of ND1 in the L3161C tumor line conferred resistance to phenformin as L3161C-ND1 cells maintained higher OXPHOS following phenformin treatment as compared to cells expressing the vector alone (FIGS. 8c-d). We then performed ¹⁸FBnTP PET imaging on L3161C-vector and L3161C-ND1 tumors treated with phenformin. ND1 expressing L3161C tumors were resistant to phenformin and showed no loss of ¹⁸FBnTP uptake as compared to vector expressing L3161C tumors (FIG. 8e). Vehicle treated L3161C-ND1 and vector tumors were both positive for ¹⁸FBnTP uptake (FIG. 8f). Our results demonstrate that ¹⁸FBnTP imaging allows for selective measurement of ΔΨ and OXPHOS in lung tumors following inhibition with multiple complex I inhibitors.

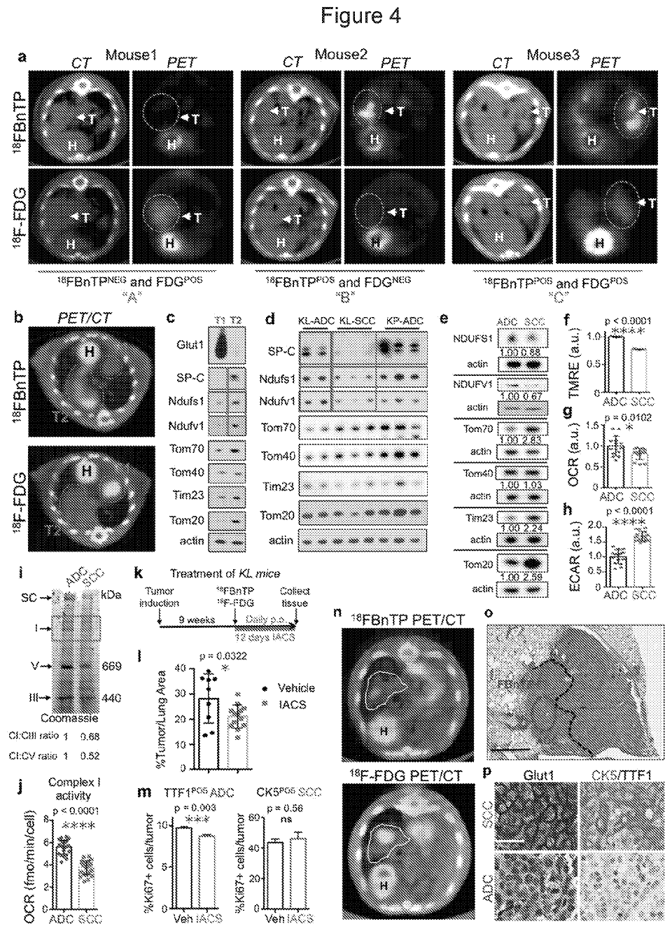

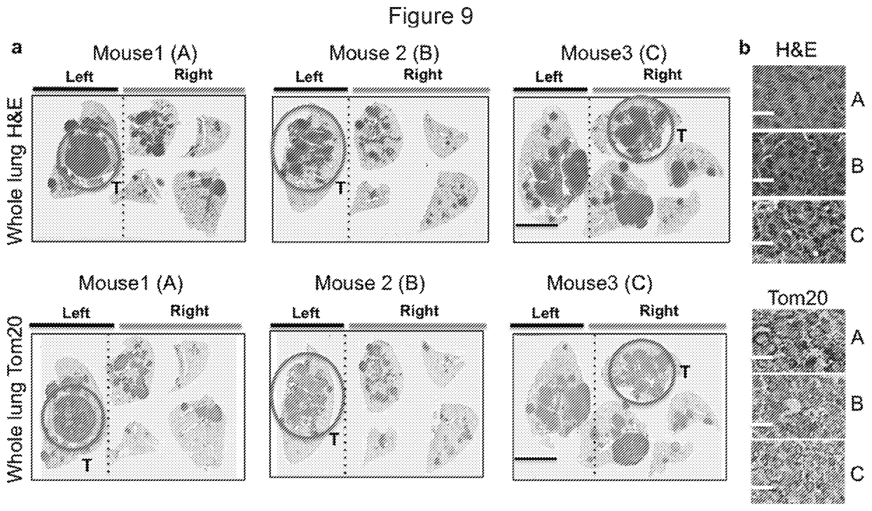

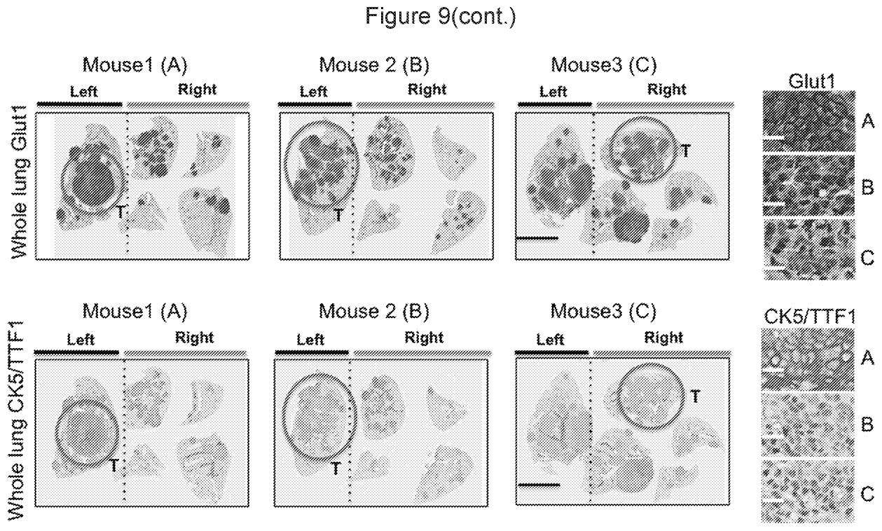

NSCLC is marked by genetic, metabolic and histological heterogeneity in tumors [Ref. 30,31,32]. We next sought to perform multi-tracer PET imaging on Kras/Lkb1 mice using both ¹⁸FBnTP and [18F]Fluoro-2-deoxyglucose (¹⁸F-FDG) PET tracers as a means to non-invasively profile glucose metabolism with ¹⁸F-FDG and mitochondrial ΔΨ with ¹⁸FBnTP. Multi-tracer PET imaging of Kras/Lkb1 lung tumors revealed distinct metabolic heterogeneity between lung tumors in which we identified three distinct tumor populations (FIG. 4a). Glycolytic tumors denoted as type “A” represent tumors positive for ¹⁸F-FDG with low uptake of ¹⁸FBnTP, tumors denoted as type “B” represent tumors positive for ¹⁸FBnTP with low uptake of ¹⁸F-FDG negative and tumors denoted as type “C” represent tumors with uptake of both ¹⁸FBnTP and ¹⁸F-FDG tracers (FIG. 4a). Histological analysis of PET imaged lung tumors revealed that type A tumors were SCC marked by positive CK5-TTF1 staining. Conversely, type B and C tumors were both positive for TTF1 and absent of CK5 staining confirming ADC histology (FIGS. 9a,b).

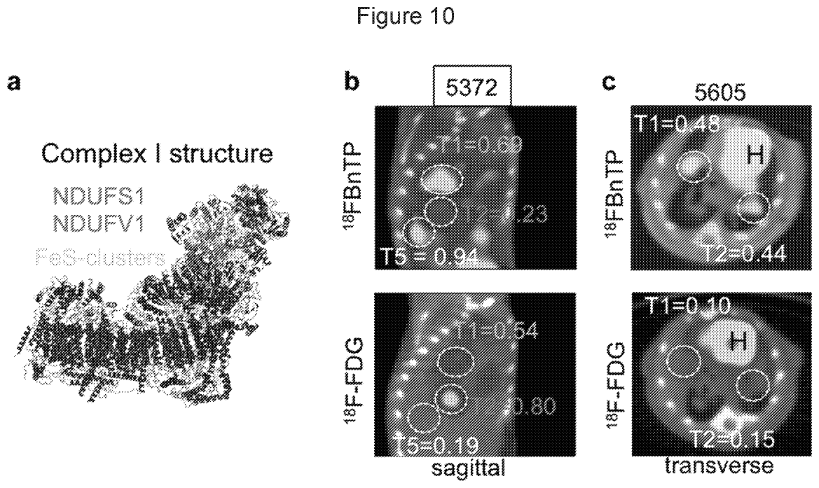

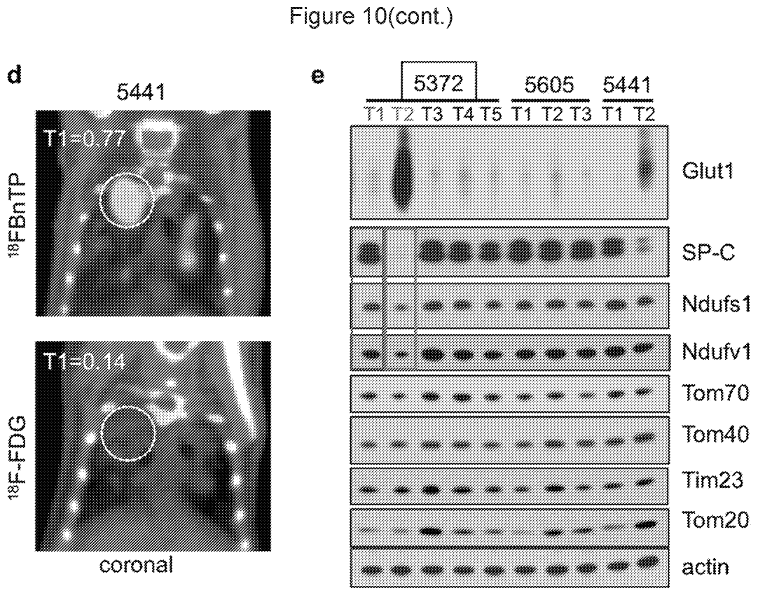

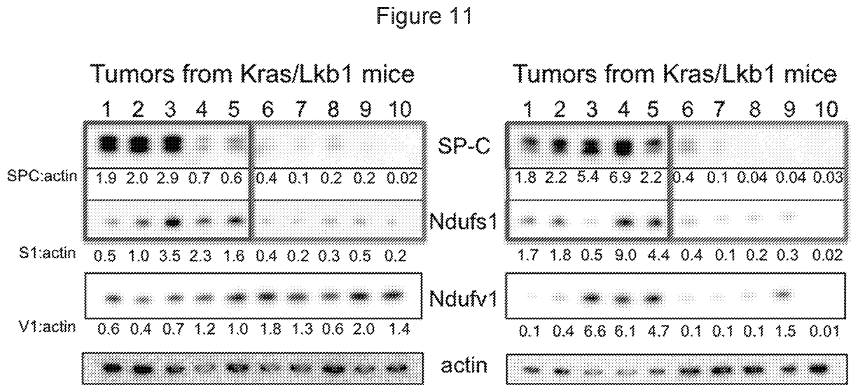

Both lung ADC and SCC showed distinct ¹⁸FBnTP and ¹⁸F-FDG profiles suggesting these tumor subtypes may have distinct bioenergetic profiles. Previous studies have shown that lung SCC tumors from Kras/Lkb1 mice are refractory to phenformin, which suggested that lung SCC tumors may harbor intrinsic defects in complex I that modulate response to complex I inhibition [Ref. 19,33]. Supporting this, a study of mitochondrial proteins in human NSCLC identified that late stage lung carcinomas have reduced expression of NDUFS1 and NDUFV1, which are complex I subunits involved in the transfer of electrons from NADH (FIG. 10a) [Ref. 34]. The authors showed that reduced NDUFS1 gene and protein expression correlated with lower overall survival of NSCLC patients. Therefore, we performed a PET guided biochemical analysis of Ndufs1 and Ndufv1 in lung ADCs and SCCs in Kras/Lkb1 mice. We identified that ¹⁸FBnTP^{NEG}/¹⁸F-FDG^{POS} SCC tumors (shown in red) had a marked reduction in Ndfus1 and Ndufv1 proteins as compared to ¹⁸FBnTP^{POS}/¹⁸F-FDG^{NEG} ADCs (shown in blue) (FIGS. 4b,c; FIGS. 10b-d). We analyzed Ndfus1 and Ndufv1 proteins in lung ADC and SCC tumors from Kras/Lkb1 mice across four independent experiments and discovered that SCCs (red boxes) had a consistent reduction in Ndfus1 as compared to ADCs (blue boxes) (FIG. 4d; FIG. 10; FIG. 11). Lung ADC and SCC were distinguished by the presence or absence of SP-C protein and immunoblots for Tom20, Tom40, Tom70 or Tim23 showed similar distribution of mitochondrial content between SCC and ADC tumors (FIGS. 4c,d; FIG. 5).

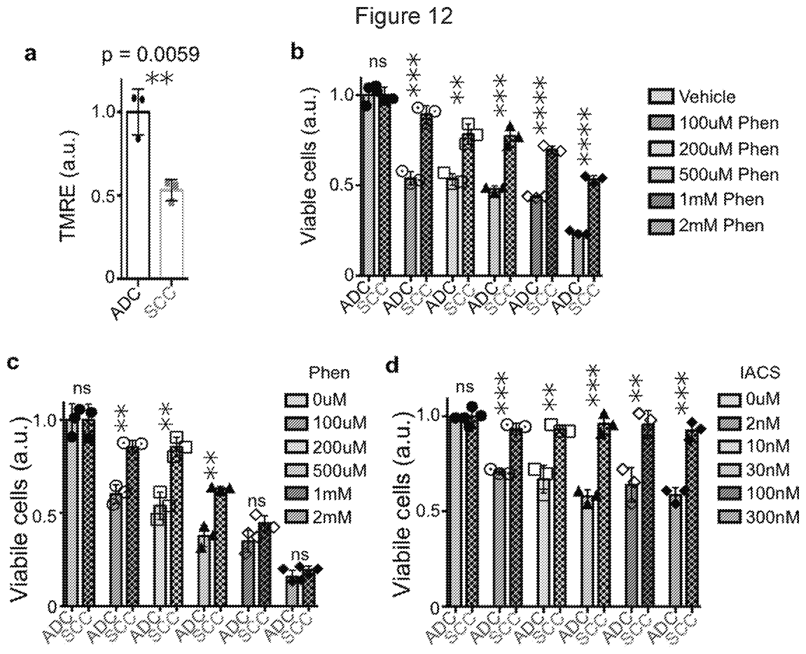

We next examined the NDUFS1 and NDUFV1 proteins in the human lung ADC cell line A549 and lung SCC line RH2. Identical to mouse lung tumors, we identified a decrease in levels of both the NDUFS1 and V1 proteins in RH2 cells compared to A549 cells (FIG. 4e). Functional analysis of mitochondrial ΔΨ in human and mouse ADC and SCC cell lines demonstrated that the SCC tumor cells had a significantly reduced ΔΨ to that of ADC cells (FIG. 4f; FIG. 12a). RH2 cells had significantly reduced oxygen consumption rate and a significantly increased extracellular acidification rate compared to A549 cells (FIGS. 4g,h). Analysis of complex I in coomassie stained blue native gels showed that RH2 cells had lower complex I levels than A549 (FIG. 4i). In addition, RH2 cells had significantly reduced complex I activity as compared to A549 cells (FIG. 4j). These results suggest that ΔΨ and complex I activity may be predictive of response to complex I inhibition—with ¹⁸FBnTP^{POS} lung ADCs predicted to have increased sensitivity compared to ¹⁸FBnTP^{NEG} lung SCCs.

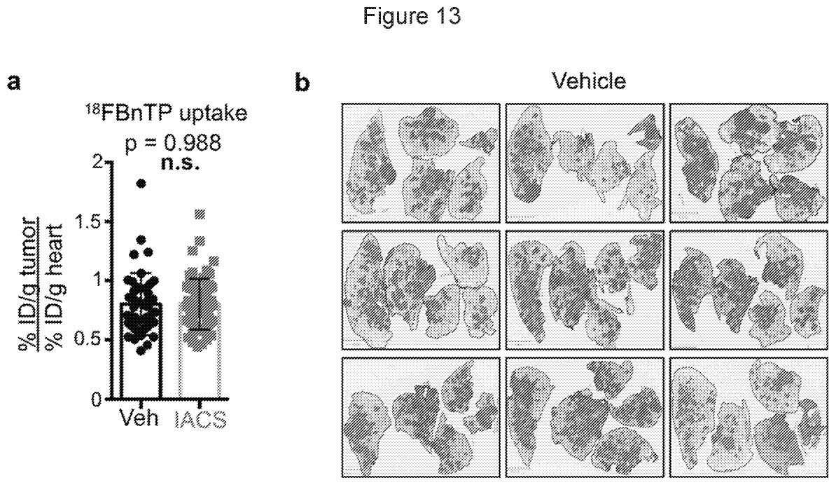

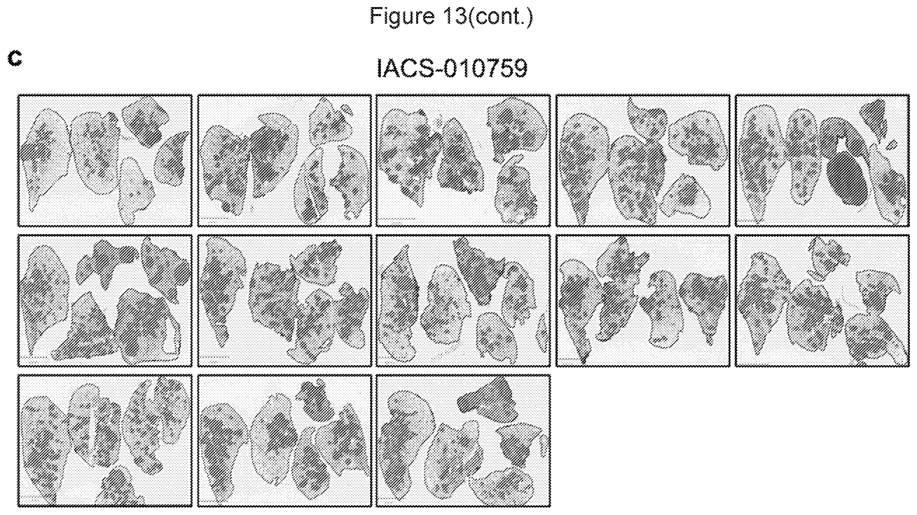

We demonstrated that both human and mouse lung ADC cell lines were more sensitive to complex I inhibitors phenformin and recently developed IACS-010759 [Ref. 35] at low doses compared to lung SCCs lines (FIGS. 12b-d). We next tested IACS-017059 in vivo on Kras/Lkb1 mice and determined that ¹⁸FBnTP^{POS} tumors were sensitive to the complex I inhibition. Kras/Lkb1 mice were imaged by ¹⁸FBnTP and ¹⁸F-FDG PET before treatment to confirm the presence of ¹⁸FBnTP^{POS} tumors and binned into equal groups based on ¹⁸FBnTP uptake in tumors. (FIG. 4k; FIG. 13a). Mice receiving IACS-010759 showed a significant reduction in tumor burden compared to those receiving vehicle (FIG. 4l; FIGS. 13b,c). Our data predicted that ¹⁸FBnTP^{POS} ADCs would show increased sensitivity to IACS-010759 compared to ¹⁸FBnTP^{NEG} SCCs. Analysis of tumor cell proliferation by Ki67 staining across ADC and SCC tumor histologies showed a significant reduction in Ki67 positive cells in TTF1+ ADCs treated with IACS-010759 while CK5+ SCCs were refractory to IACS-010759 (FIG. 4m).

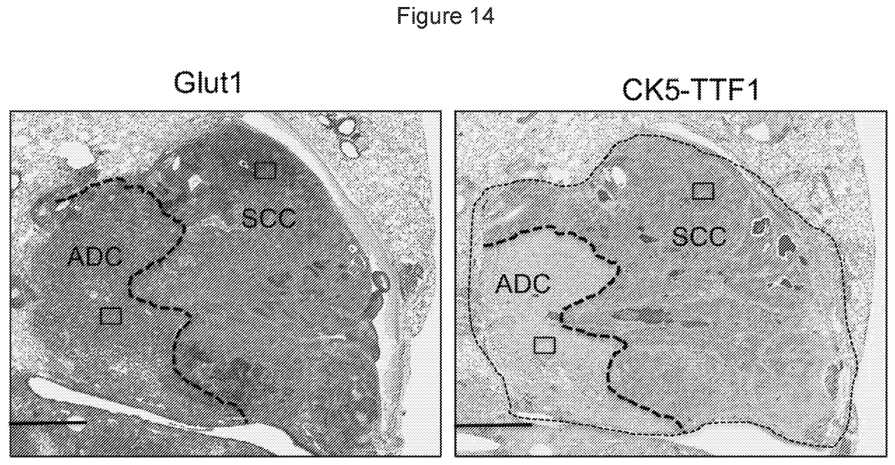

Spurred by the ability to use multi-tracer PET imaging to detect metabolic heterogeneity across multiple tumors, we next asked whether ¹⁸FBnTP and ¹⁸F-FDG PET imaging could detect metabolic heterogeneity within an individual lung tumor. We identified heterogeneous uptake of both ¹⁸FBnTP and ¹⁸F-FDG in lung nodules from Kras/Lkb1 mice as represented in FIGS. 4n. Histological analysis of the tumor revealed that the ¹⁸F-FDG avid region was positive for the SCC tumor marker CK5 and had elevated expression of Glut1 thus explaining the elevated ¹⁸F-FDG uptake. In contrast, the ¹⁸FBnTP avid region that stained positive for the ADC marker TTF1 and negative for CK5 had low expression of Glut1 thus explaining the low ¹⁸F-FDG uptake (FIG. 4o,p; FIG. 14). These results demonstrate that multi-tracer PET imaging with ¹⁸FBnTP and ¹⁸F-FDG was able to identify distinct mitochondrial and metabolic heterogeneity within individual lung tumors. Our study represents a novel and non-invasive approach to using ¹⁸FBnTP PET imaging to profile mitochondrial ΔΨ and functional mitochondrial heterogeneity within NSCLC. We detected distinct mitochondrial ΔΨ and complex I activity profiles in lung SCC and ADCs tumors that were predictive of response to phenformin. With complex I inhibitors such as IACS-010759 poised to enter testing in clinical trials [Ref. 35], our results suggest ¹⁸FBnTP may function as a noninvasive biomarker to guide the delivery of complex I inhibitors. ¹⁸FBnTP PET imaging represents a valuable resource not only to the field of cancer metabolism but one that can be extended to other fields actively investigating mitochondrial activity in aging, physiology and disease.

Thus, our invention discloses the novel use of the positron emission tomography (PET) tracer ¹⁸FBnTP as a companion diagnostic to guide the delivery of mitochondrial complex I inhibitors and other small molecules that inhibit the electron transport chain (ETC) and reduce mitochondrial membrane potential and oxidative phosphorylation (OXPHOS). ¹⁸FBnTP PET is able to measure mitochondrial membrane potential (ΔΨ) and complex I and II activity in lung tumors. Lung tumors with high uptake of the ¹⁸FBnTP tracer are dependent on mitochondrial complex I activity and thus sensitive to small molecule complex I inhibitors. Using ¹⁸FBnTP PET imaging, we are able to successfully identify lung tumors that are sensitive to complex I inhibitors such as metformin, phenformin, IACS-01759 and rotenone. Without wishing to be bound to theory, tumors may be sensitive to a broad number of complex I inhibitors such as IACS-01759 and mubitrinib (TAK-165) and other like small molecules inhibitors of ΔΨ and OXPHOS.

The invention also discloses the use of ¹⁸FBnTP PET imaging on cancer patients to identify tumors with complex I and II-dependent metabolism so that they can be precisely treated using complex I and/or II inhibitors. Additionally, the ¹⁸FBnTP PET tracer can be used to detect increases in the ETC activity and OXPHOS resulting in increases in the mitochondrial membrane potential following treatment with small molecule compounds such as oligomycin.

### REFERENCES

1 Mitchell, P. & Moyle, J. Evidence discriminating between the chemical and the chemiosmotic mechanisms of electron transport phosphorylation. Nature 208, 1205-1206 (1965).
2 Mitchell, P. & Moyle, J. Stoichiometry of proton translocation through the respiratory chain and adenosine triphosphatase systems of rat liver mitochondria. Nature 208, 147-151 (1965).
3 Morais, R. et al. Tumor-forming ability in athymic nude mice of human cell lines devoid of mitochondrial DNA. Cancer Res 54, 3889-3896 (1994).
4 Desjardins, P., Frost, E. & Morais, R. Ethidium bromide-induced loss of mitochondrial DNA from primary chicken embryo fibroblasts. Mol Cell Biol 5, 1163-1169 (1985).
5 Cavalli, L. R., Varella-Garcia, M. & Liang, B. C. Diminished tumorigenic phenotype after depletion of mitochondrial DNA. Cell Growth Differ 8, 1189-1198 (1997).
6 Madar, I. et al. Characterization of uptake of the new PET imaging compound 18F-fluorobenzyl triphenyl phosphonium in dog myocardium. J Nucl Med 47, 1359-1366 (2006).
7 Weinberg, F. et al. Mitochondrial metabolism and ROS generation are essential for Kras-mediated tumorigenicity. Proc Natl Acad Sci U S A 107, 8788-8793, doi:1003428107 [pii] 10.1073/pnas.1003428107 (2010).
8 Ji, H. et al. LKB1 modulates lung cancer differentiation and metastasis. Nature 448, 807-810 (2007).
9 Shackelford, D. B. et al. LKB1 inactivation dictates therapeutic response of non-small cell lung cancer to the metabolism drug phenformin. Cancer Cell 23, 143-158, doi:10.1016/j.ccr.2012.12.008 (2013).
10 Madar, I. et al. Characterization of membrane potential-dependent uptake of the novel PET tracer 18F-fluorobenzyl triphenylphosphonium cation. Eur J Nucl Med Mol Imaging 34, 2057-2065, doi:10.1007/s00259-007-0500-8 (2007).
11 Smith, R. A., Hartley, R. C. & Murphy, M. P. Mitochondria-targeted small molecule therapeutics and probes. Antioxidants & redox signaling 15, 3021-3038, doi:10.1089/ars.2011.3969 (2011).
12 Kim, D. Y. et al. Evaluation of a mitochondrial voltage sensor, (18F-fluoropentyl)triphenylphosphonium cation, in a rat myocardial infarction model. J Nucl Med 53, 1779-1785, doi:10.2967/jnumed.111.102657 (2012).
13 Madar, I. et al. Detection and quantification of the evolution dynamics of apoptosis using the PET voltage sensor 18F-fluorobenzyl triphenyl phosphonium. J Nucl Med 50, 774-780, doi:10.2967/jnumed.108.061283 (2009).
14 Zhao, G. et al. Membrane potential-dependent uptake of 18F-triphenylphosphonium--a new voltage sensor as an imaging agent for detecting burn-induced apoptosis. J Surg Res 188, 473-479, doi:10.1016/j.jss.2014.01.011 (2014).
15 Logan, A. et al. Assessing the Mitochondrial Membrane Potential in Cells and In Vivo using Targeted Click Chemistry and Mass Spectrometry. Cell Metab 23, 379-385, doi:10.1016/j.cmet.2015.11.014 (2016).
16 Waldmann, C. M. et al. An Automated Multidose Synthesis of the Potentiometric PET Probe 4-[(18)F]Fluorobenzyl-Triphenylphosphonium ([(18)F]FBnTP). Mol Imaging Biol, doi:10.1007/s11307-017-1119-1 (2017).
17 Kroemer, G., Galluzzi, L. & Brenner, C. Mitochondrial membrane permeabilization in cell death. Physiological reviews 87, 99-163, doi:10.1152/physrev.00013.2006 (2007).
18 Dykens, J. A. et al. Biguanide-induced mitochondrial dysfunction yields increased lactate production and cytotoxicity of aerobically-poised HepG2 cells and human hepatocytes in vitro. Toxicol Appl Pharmacol 233, 203-210, doi:50041-008X(08)00361-X [pi i] 10.1016/j.taap.2008.08.013 (2008).
19 Li, F. et al. LKB1 Inactivation Elicits a Redox Imbalance to Modulate Non-small Cell Lung Cancer Plasticity and Therapeutic Response. Cancer Cell 27, 698-711, doi:10.1016/j.cce11.2015.04.001 (2015).
20 Giordano, S., Lee, J., Darley-Usmar, V. M. & Zhang, J. Distinct effects of rotenone, 1-methyl-4-phenylpyridinium and 6-hydroxydopamine on cellular bioenergetics and cell death. PLoS One 7, e44610, doi:10.1371/journal.pone.0044610 (2012).
21 Singer, T. P. & Ramsay, R. R. The reaction sites of rotenone and ubiquinone with mitochondrial NADH dehydrogenase. Biochim Biophys Acta 1187, 198-202 (1994).
22 Caboni, P. et al. Rotenone, deguelin, their metabolites, and the rat model of Parkinson's disease. Chem Res Toxicol 17, 1540-1548, doi:10.1021/tx049867r (2004).
23 Bridges, H. R., Jones, A. J., Pollak, M. N. & Hirst, J. Effects of metformin and other biguanides on oxidative phosphorylation in mitochondria. Biochem J 462, 475-487, doi:10.1042/BJ20140620 (2014).
24 El-Mir, M. Y. et al. Dimethylbiguanide inhibits cell respiration via an indirect effect targeted on the respiratory chain complex I. J Biol Chem 275, 223-228 (2000).
25 Owen, M. R., Doran, E. & Halestrap, A. P. Evidence that metformin exerts its anti-diabetic effects through inhibition of complex 1 of the mitochondrial respiratory chain. Biochem J 348 Pt 3, 607-614 (2000).
26 Wheaton, W. W. et al. Metformin inhibits mitochondrial complex I of cancer cells to reduce tumorigenesis. Elife 3, e02242, doi:10.7554/eLife.02242 (2014).
27 Bridges, H. R., Sirvio, V. A., Agip, A. N. & Hirst, J. Molecular features of biguanides required for targeting of mitochondrial respiratory complex I and activation of AMP-kinase. BMC Biol 14, 65, doi:10.1186/s12915-016-0287-9 (2016).
28 Sanchez-Rangel, E. & Inzucchi, S. E. Metformin: clinical use in type 2 diabetes. Diabetologia 60, 1586-1593, doi:10.1007/s00125-017-4336-x (2017).
29 Birsoy, K. et al. Metabolic determinants of cancer cell sensitivity to glucose limitation and biguanides. Nature 508, 108-112, doi:10.1038/nature13110 (2014).
30 Hensley, C. T. et al. Metabolic Heterogeneity in Human Lung Tumors. Cell 164, 681-694, doi:10.1016/j.ce11.2015.12.034 (2016).
31 Kerr, K. M. Personalized medicine for lung cancer: new challenges for pathology. Histopathology 60, 531-546, doi:10.1111/j.1365-2559.2011.03854.x (2012).
32 de Bruin, E. C. et al. Spatial and temporal diversity in genomic instability processes defines lung cancer evolution. Science 346, 251-256, doi:10.1126/science.1253462 (2014).
33 Momcilovic, M. et al. Heightening Energetic Stress Selectively Targets LKB1-Deficient Non-Small Cell Lung Cancers. Cancer Res 75, 4910-4922, doi: 10.1158/0008-5472. CAN-15-0797 (2015).
34 Su, C. Y., Chang, Y. C., Yang, C. J., Huang, M. S. & Hsiao, M. The opposite prognostic effect of NDUFS1 and NDUFS8 in lung cancer reflects the oncojanus role of mitochondrial complex I. Sci Rep 6, 31357, doi:10.1038/srep31357 (2016).
35 Molina, J. R. et al. An inhibitor of oxidative phosphorylation exploits cancer vulnerability. Nat Med 24, 1036-1046, doi:10.1038/s41591-018-0052-4 (2018).
36 Gilbert-Ross, M. et al. Targeting adhesion signaling in KRAS, LKB1 mutant lung adenocarcinoma. JCI Insight 2, e90487, doi:10.1172/jci.insight.90487 (2017).
37 Poczobutt, J. M. et al. Expression Profiling of Macrophages Reveals Multiple Populations with Distinct Biological Roles in an Immunocompetent Orthotopic Model of Lung Cancer. J Immunol 196, 2847-2859, doi:10.4049/jimmuno1.1502364 (2016).
38 Momcilovic, M. et al. The GSK3 Signaling Axis Regulates Adaptive Glutamine Metabolism in Lung Squamous Cell Carcinoma. Cancer Cell 33, 905-921 e905, doi:10.1016/j.cce11.2018.04.002 (2018).
39 Wittig, I., Braun, H. P. & Schagger, H. Blue native PAGE. Nat Protoc 1, 418-428, doi:10.1038/nprot.2006.62 (2006).

The citation of any document is not to be construed as an admission that it is prior art with respect to the present invention.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the embodiments contained herein.

What is claimed is:

1. A method for detecting or ruling out non-small cell lung cancer (NSCLC) in a patient, the method comprising:
(a) administering to a patient a detectable amount of a compound of formula (I): 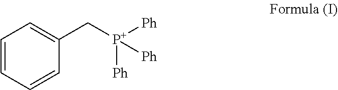 [SMILES: c1ccc(C[P+](c2ccccc2)(c2ccccc2)c2ccccc2)cc1]
wherein the compound is targeted to any NSCLC tumor in the patient; and
(b) acquiring an image to detect the presence or absence of any NSCLC tumor in the patient,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

2. The method of claim 1 wherein:
step (b) comprises acquiring the image using an imaging technique selected from positron emission tomography imaging, positron emission tomography with computed tomography imaging, or positron emission tomography with magnetic resonance imaging.

3. The method of claim 1 wherein:
at least one of the atoms in formula (I) is replaced with ¹⁸F.

4. A method for evaluating mitochondrial complex I inhibition of a non-small cell lung cancer (NSCLC) in a subject, the method comprising:
(a) administering an effective amount of a mitochondrial complex I inhibitor;
(b) administering a detectable amount of a compound of formula (I): 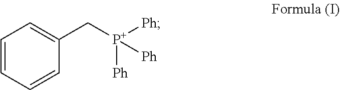 [SMILES: c1ccc(C[P+](c2ccccc2)(c2ccccc2)c2ccccc2)cc1]
(c) waiting a time sufficient to allow the compound to accumulate at a tissue or cell site to be imaged; and
(d) imaging the cells or tissues with an imaging technique,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

5. The method of claim 4 wherein:
step (d) comprises acquiring the image using an imaging technique selected from positron emission tomography imaging, positron emission tomography with computed tomography imaging, or positron emission tomography with magnetic resonance imaging.

6. The method of claim 4 wherein:
the mitochondrial complex I inhibitor is selected from metformin, phenformin, or rotenone.

7. The method of claim 4 wherein:
at least one of the atoms in formula (I) is replaced with ¹⁸F.

8. A method for evaluating mitochondrial complex V inhibition of a non-small cell lung cancer (NSCLC) in a subject, the method comprising:
(a) administering an effective amount of a mitochondrial complex V inhibitor;
(b) administering a detectable amount of a compound of formula (I): 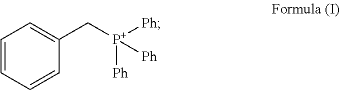 [SMILES: c1ccc(C[P+](c2ccccc2)(c2ccccc2)c2ccccc2)cc1]
(c) waiting a time sufficient to allow the compound to accumulate at a tissue or cell site to be imaged; and
(d) imaging the cells or tissues with an imaging technique,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

9. The method of claim 8 wherein:
step (d) comprises acquiring the image using an imaging technique selected from positron emission tomography imaging, positron emission tomography with computed tomography imaging, or positron emission tomography with magnetic resonance imaging.

10. The method of claim 8 wherein:
the mitochondrial complex V inhibitor is oligomycin.

11. The method of claim 8 wherein:
at least one of the atoms in formula (I) is replaced with ¹⁸F.

12. A method for evaluating mitochondrial membrane potential gradient (ΔΨ) in NSCLC in a subject, the method comprising:
(a) administering a detectable amount of a compound of formula (I): 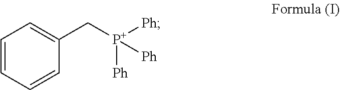 [SMILES: c1ccc(C[P+](c2ccccc2)(c2ccccc2)c2ccccc2)cc1]
and
(b) acquiring an image to detect the presence or absence of formula (I) in a NSCLC tumor in the subject,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

13. The method of claim 12 wherein:
step (b) comprises acquiring the image using an imaging technique selected from positron emission tomography imaging, positron emission tomography with computed tomography imaging, or positron emission tomography with magnetic resonance imaging.

14. The method of claim 12 wherein:
at least one of the atoms in formula (I) is replaced with ¹⁸F.

15. A method for evaluating mitochondrial membrane potential gradient (ΔΨ) in NSCLC in a subject, the method comprising:
(a) administering a detectable amount of a compound of formula (I): 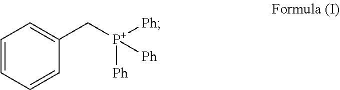 [SMILES: c1ccc(C[P+](c2ccccc2)(c2ccccc2)c2ccccc2)cc1]
and
(b) acquiring an image to detect the presence or absence of formula (I) in a NSCLC tumor in the subject,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.
(c) administering an effective amount of a mitochondrial complex I inhibitor;
(d) acquiring an image to detect the presence or absence of formula (I) in a NSCLC tumor in the subject.

16. The method of claim 15 wherein:
steps (b) and (d) comprise acquiring the image using an imaging technique selected from positron emission tomography imaging, positron emission tomography with computed tomography imaging, or positron emission tomography with magnetic resonance imaging.

17. The method of claim 16 wherein:
the mitochondrial complex I inhibitor is selected from metformin, phenformin, or rotenone.

18. The method of claim 16 wherein:
at least one of the atoms in formula (I) is replaced with ¹⁸F.

19. A method for evaluating mitochondrial complex I activity and mitochondrial membrane potential gradient (ΔΨ) in NSCLC, the method comprising:
(a) administering a detectable amount of a compound of formula (I): 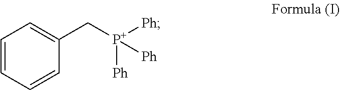 [SMILES: c1ccc(C[P+](c2ccccc2)(c2ccccc2)c2ccccc2)cc1]
and
(b) acquiring an image to detect the presence or absence of formula (I) in a NSCLC tumor in the subject,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

20. The method of claim 19 wherein:
step (b) comprises acquiring the image using an imaging technique selected from positron emission tomography imaging, positron emission tomography with computed tomography imaging, or positron emission tomography with magnetic resonance imaging.

21. The method of claim 19 wherein:
at least one of the atoms in formula (I) is replaced with ¹⁸F.

22. A method for detecting mitochondrial and metabolic heterogeneity within individual lung tumors in a subject, the method comprising:
(a) administering a detectable amount of a compound of formula (I): 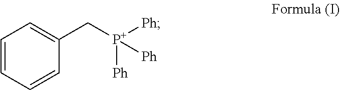 [SMILES: c1ccc(C[P+](c2ccccc2)(c2ccccc2)c2ccccc2)cc1]
(b) administering a detectable amount of a compound of formula (II) 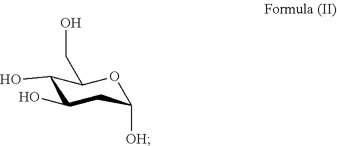
and
(c) acquiring an image to detect the presence or absence of formula (I) and formula (II) in a NSCLC tumor in the subject,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.
wherein at least one of the atoms in formula (II) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

23. The method of claim 22 wherein:
step (c) comprises acquiring the image using an imaging technique selected from positron emission tomography imaging, positron emission tomography with computed tomography imaging, or positron emission tomography with magnetic resonance imaging.

24. The method of claim 22 wherein:
at least one of the atoms in formula (I) is replaced with ¹⁸F, and
at least one of the atoms in formula (II) is replaced with ¹⁸F.

25. A method for the treatment of lung adenocarcinoma (ADC) in a subject, the method comprising:
(a) administering a detectable amount of a compound of formula (I): 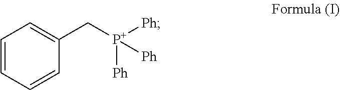 [SMILES: c1ccc(C[P+](c2ccccc2)(c2ccccc2)c2ccccc2)cc1]
(b) acquiring an image to detect the presence of an ADC tumor in the subject; and
(c) administering an effective amount of a mitochondrial complex I inhibitor,.
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

26. The method of claim 25 wherein:
the mitochondrial complex I inhibitor is selected from metformin, phenformin, rotenone, or IACS-010759.

27. The method of claim 25 wherein:
step (d) comprises acquiring the image using an imaging technique selected from positron emission tomography imaging, positron emission tomography with computed tomography imaging, or positron emission tomography with magnetic resonance imaging.

28. The method of claim 25 wherein:
at least one of the atoms in formula (I) is replaced with ¹⁸F.

29. A method for the treatment of lung adenocarcinoma (ADC) in a subject, the method comprising:
(a) administering a detectable amount of a compound of formula (I): 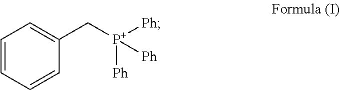 [SMILES: c1ccc(C[P+](c2ccccc2)(c2ccccc2)c2ccccc2)cc1]
(b) acquiring an image to detect the presence of a Thyroid transcription factor 1 (TTF1)+ADC tumor in the subject; and
(c) administering an effective amount of a mitochondrial complex I inhibitor,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

30. The method of claim 29 wherein:
the mitochondrial complex I inhibitor is selected from metformin, phenformin, rotenone, or IACS-010759.

31. The method of claim 29 wherein:
step (b) comprises acquiring the image using an imaging technique selected from positron emission tomography imaging, positron emission tomography with computed tomography imaging, or positron emission tomography with magnetic resonance imaging.

32. The method of claim 29 wherein:
at least one of the atoms in formula (I) is replaced with ¹⁸F.

33. A method for evaluating mitochondrial complex II activity and mitochondrial membrane potential gradient (ΔΨ) in NSCLC, the method comprising:
(a) administering a detectable amount of a compound of formula (I): 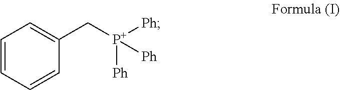 [SMILES: c1ccc(C[P+](c2ccccc2)(c2ccccc2)c2ccccc2)cc1]
and
(b) acquiring an image to detect the presence or absence of formula (I) in a NSCLC tumor in the subject,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

34. The method of claim 33 wherein:
step (b) comprises acquiring the image using an imaging technique selected from positron emission tomography imaging, positron emission tomography with computed tomography imaging, or positron emission tomography with magnetic resonance imaging.

35. The method of claim 33 wherein:
at least one of the atoms in formula (I) is replaced with ¹⁸F.

36. A method for evaluating electron transport chain (ETC) and oxidative phosphorylation (OXPHOS) of a non-small cell lung cancer (NSCLC) in a subject, the method comprising:
(a) administering an effective amount of a small molecule;
(b) administering a detectable amount of a compound of formula (I): 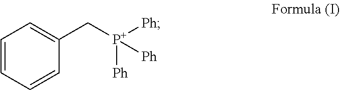 [SMILES: c1ccc(C[P+](c2ccccc2)(c2ccccc2)c2ccccc2)cc1]
(c) waiting a time sufficient to allow the compound to accumulate at a tissue or cell site to be imaged; and
(d) imaging the cells or tissues with an imaging technique,
wherein at least one of the atoms in formula (I) is replaced with ¹¹C, ¹³N, ¹⁵O, ¹⁸F, ^{34m}CI, ³⁸K, ⁴⁵Ti, ⁵¹Mn, ^{52m}Mn, ⁵²Fe, ⁵⁵Co, ⁶⁰CU, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

37. The method of claim 36 wherein:
step (d) comprises acquiring the image using an imaging technique selected from positron emission tomography imaging, positron emission tomography with computed tomography imaging, or positron emission tomography with magnetic resonance imaging.

38. The method of claim 36 wherein the mitochondrial membrane potential increases after step (a).

39. The method of claim 36 wherein:
the small molecule is oligomycin.

40. The method of claim 36 wherein:
at least one of the atoms in formula (I) is replaced with ¹⁸F.
